(19) United States
(12) Patent Application Publication
(10) Pub. No.: US 2026/0249038 A1
(43) Pub. Date: Aug. 27, 2026

(54) SYSTEM

(71) Applicant: SoftBank Group Corp., Tokyo (JP)

(72) Inventor: Chie HIGASHI, Tokyo (JP)

(73) Assignee: SoftBank Group Corp., Tokyo (JP)

(21) Appl. No.: 19/543,867

(22) Filed: Feb. 19, 2026

(30) Foreign Application Priority Data
Feb. 21, 2025 (JP) 2025-027007

(51) Int. Cl. A61M 21/02; A61B 5/00; A61B 5/0205; A61B 5/021; A61B 5/024; A61B 5/11; A61B 5/16; A61M 21/00; G06F 40/166; G10L 13/027; G10L 13/10; G10L 15/16; G10L 25/63; G16H 40/67

(52) U.S. Cl. CPC A61M 21/02; A61B 5/0002; A61B 5/02055; A61B 5/165; A61B 5/4839; A61B 5/725; A61B 5/7253; A61B 5/7267; A61B 5/7278; A61B 5/7282; A61B 5/746; G06F 40/166; G10L 13/027; G10L 13/10; G10L 15/16; G10L 25/63; G16H 40/67; A61B 5/021; A61B 5/024; A61B 5/1126; A61B 5/4803; A61B 2560/0242; A61B 2562/0219; A61M 2021/0027

(57) ABSTRACT

The system according to the embodiment comprises a verbalization unit, a response unit, an output unit, a provision unit, a monitoring unit, and a notification unit. The verbalization unit verbalizes spoken content using speech recognition technology. The response unit generates a response based on the content verbalized by the verbalization unit and responds by voice. The output unit outputs the response generated by the response unit by voice. The provision unit provides the voice output by the output unit to an elderly person. The monitoring unit monitors the health condition of the elderly person based on the voice provided by the provision unit. The notification unit notifies a medical institution or family in case of emergency based on the health condition monitored by the monitoring unit.

### CROSS-REFERENCE TO RELATED APPLICATION

The present application claims priority to and incorporates by reference the entire contents of Japanese Patent Application No. 2025-027007 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, systems that both alleviate the loneliness of elderly persons and provide appropriate responses in emergencies have not been sufficiently provided, and there is room for improvement.

### SUMMARY OF THE INVENTION

The system according to the embodiment comprises a verbalization unit, a response unit, an output unit, a provision unit, a monitoring unit, and a notification unit. The verbalization unit verbalizes spoken content using speech recognition technology. The response unit generates a response based on the content verbalized by the verbalization unit and responds by voice. The output unit outputs the response generated by the response unit by voice. The provision unit provides the voice output by the output unit to an elderly person. The monitoring unit monitors the health condition of the elderly person based on the voice provided by the provision unit. The notification unit notifies a medical institution or family in case of emergency based on the health condition monitored by the monitoring unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

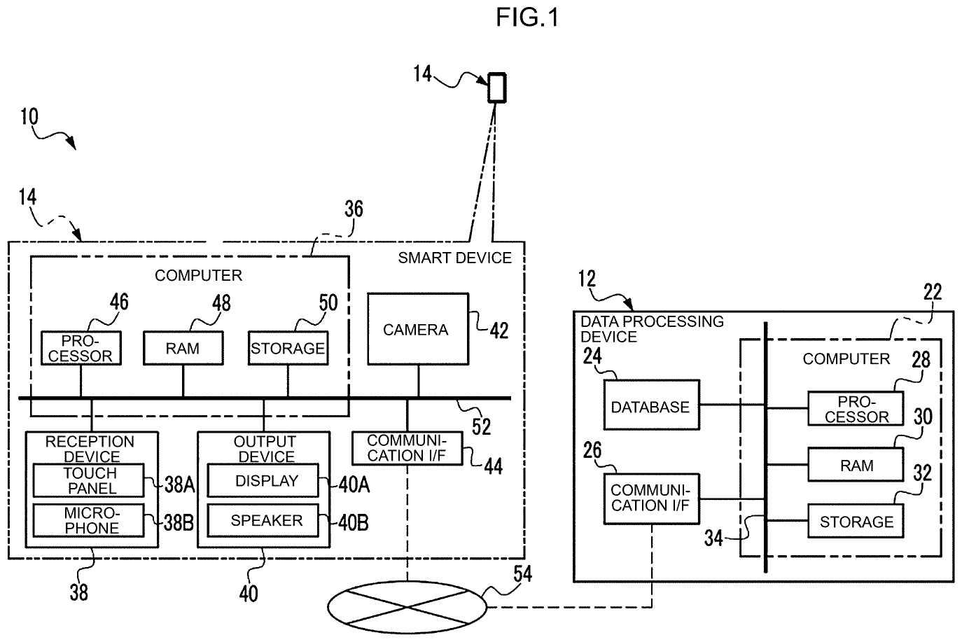

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

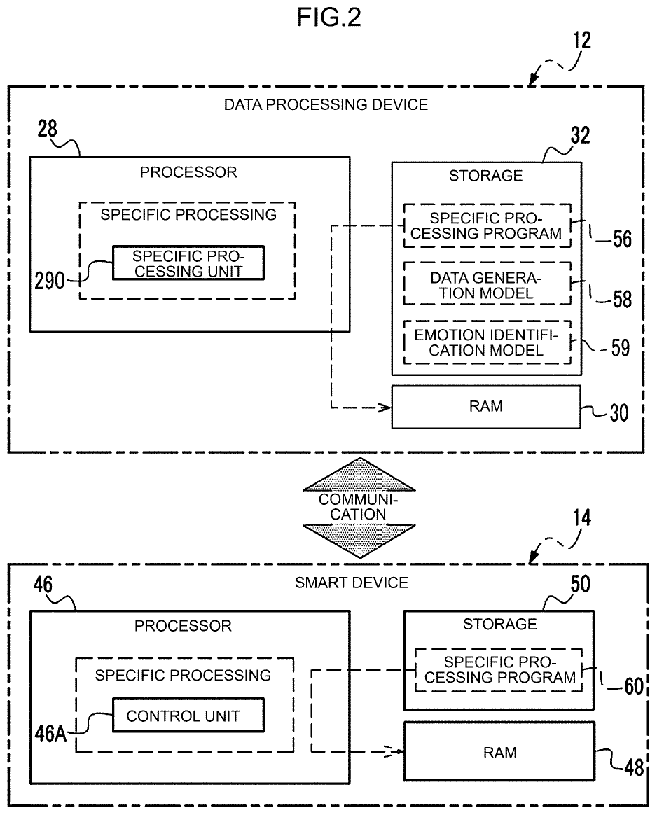

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The family AI robot system according to the embodiment of the present invention is a system designed for elderly persons living alone. This system has the function of verbalizing spoken content using speech recognition technology and responding by voice. As a result, it can alleviate the sense of loneliness experienced by elderly persons. In addition, it is equipped with a function to notify medical institutions or family members in case of emergency. The robot is cat-shaped, making it a friendly design for elderly persons. For example, when an elderly person speaks to the robot, the robot verbalizes the content using speech recognition technology. Next, the robot generates an appropriate response based on the verbalized content and responds by voice. For instance, if the elderly person says, “The weather is nice today,” the robot responds, “Yes, it is sunny today.” Furthermore, the robot has a function to monitor the health condition of the elderly person. For example, the robot periodically measures the heart rate and body temperature of the elderly person, and if any abnormality is detected, it notifies medical institutions or family members. The notification is performed automatically by the robot, so the elderly person does not need to notify by themselves. This robot not only alleviates the sense of loneliness of elderly persons in an aging society but also enables emergency response, thereby improving the quality of life for elderly persons. Thus, the family AI robot system can alleviate the sense of loneliness of elderly persons and enable emergency response. Specifically, the family AI robot system is equipped with multiple computer modules, including a speech recognition unit, a natural language processing unit, a speech synthesis unit, a health monitoring unit, a notification unit, a dialogue history management unit, and an emotion estimation unit. The speech recognition unit uses deep neural networks (e.g., convolutional neural networks, recurrent neural networks, or Transformer-based speech recognition models) to convert input speech waveforms (e.g., 16 kHz sampled one-dimensional time-series tensors, length 16,000 to 32,000 samples, examples: “The weather is nice today,” “I'm hungry”) into feature quantities such as mel spectrograms, and then into phoneme sequences or character sequences. Examples of input include one second of speech such as “Hello,” or three seconds of speech such as “I want to go to the hospital today.” The output of the speech recognition unit is string data (e.g., “The weather is nice today,” “I'm hungry”), which is passed to the natural language processing unit. The natural language processing unit uses large language models or rule-based dialogue management algorithms to extract intent (e.g., “conversation about the weather,” “request for food”) and estimate emotion (e.g., “joy,” “sadness”) from the input text, and generates response candidates (e.g., “It is sunny today,” “Would you like something to eat?”) in the response generation unit. The response generation unit uses template-based or generative models (e.g., Transformer-based text generation models) to generate response sentences. The generated response sentences are passed to the speech synthesis unit, which uses neural speech synthesis models such as WaveNet or Tacotron to convert text (e.g., “It is sunny today”) into speech waveforms (e.g., 16 kHz, PCM data). Examples of output from the speech synthesis unit include a gentle female voice saying “It is sunny today,” or a calm male voice saying “Would you like something to eat?” The health monitoring unit collects biosignals obtained from heart rate sensors or body temperature sensors (e.g., heart rate data every minute, body temperature data of 36.5° C.) as time-series vectors, and detects values outside the normal range (e.g., heart rate exceeding 120 bpm, body temperature exceeding 38.0° C.) using anomaly detection algorithms (e.g., threshold judgment, time-series anomaly detection models). If an abnormality is detected, the notification unit automatically sends notification messages (e.g., “The heart rate of the elderly person has exceeded 120 bpm,” “The body temperature is 38.2° C.”) to family members or medical institutions via SMS or the Internet. These processes are realized by a series of unconventional computer processing flows, including automatic collection of sensor data, AI-based anomaly judgment, and automatic notification, which differ from conventional manual monitoring or telephone contact by humans, resulting in technical effects such as reduction of false detections, minimization of notification delays, and centralized management of data. Furthermore, the dialogue history management unit accumulates past conversation content and health condition data as a time-series database, which can be used to generate individually optimized dialogues and health advice. The emotion estimation unit estimates the emotional state of the elderly person from speech features (e.g., F0, energy, speech rate) and text content, and dynamically adjusts the tone, speed, and content of responses and speech synthesis. As a result, not only automatic responses but also personalized dialogue, health management, and emergency response tailored to the individual state and emotions of each elderly person become possible, greatly enhancing the technical value of the AI robot system. Application fields include monitoring of elderly persons living alone, remote medical support, automatic dialogue and health management in nursing care facilities, and support for persons with disabilities.

The family AI robot system according to the embodiment comprises a verbalization unit, a response unit, an output unit, a provision unit, a monitoring unit, and a notification unit. The verbalization unit verbalizes spoken content using speech recognition technology. For example, the verbalization unit can accurately verbalize the spoken content of an elderly person using deep learning-based speech recognition technology. The verbalization unit may also use HMM (Hidden Markov Model)-based speech recognition technology. The response unit generates an appropriate response based on the content verbalized by the verbalization unit. For example, the response unit can generate an appropriate response to the elderly person using natural language generation technology. The response unit may also use template-based response generation technology. The output unit outputs the response generated by the response unit by voice. For example, the output unit can output the generated response as high-quality voice using speech synthesis technology. The provision unit provides the voice output by the output unit to the elderly person. For example, the provision unit can provide the output voice to the elderly person at a volume that is easy to hear. The monitoring unit monitors the health condition of the elderly person based on the voice provided by the provision unit. For example, the monitoring unit can periodically measure the heart rate and body temperature of the elderly person and monitor the health condition. The notification unit notifies a medical institution or family in case of emergency based on the health condition monitored by the monitoring unit. For example, the notification unit can automatically notify a medical institution or family when an abnormality is detected. As a result, the family AI robot system according to the embodiment can alleviate the sense of loneliness of elderly persons and enable emergency response. Specifically, the family AI robot system implements each unit as multiple computer modules, and the speech recognition unit uses convolutional neural networks, recurrent neural networks, or Transformer-based speech recognition models to convert input speech waveforms (e.g., 16 kHz sampled one-dimensional time-series tensors, length 16,000 to 32,000 samples, examples: “The weather is nice today,” “I'm hungry”) into feature quantities such as mel spectrograms, and then into phoneme sequences or character sequences. Examples of input include one second of speech such as “Hello,” or three seconds of speech such as “I want to go to the hospital today.” The output of the speech recognition unit is string data (e.g., “The weather is nice today,” “I'm hungry”), which is passed to the natural language processing unit. The natural language processing unit uses large language models or rule-based dialogue management algorithms to extract intent (e.g., “conversation about the weather,” “request for food”) and estimate emotion (e.g., “joy,” “sadness”) from the input text, and generates response candidates (e.g., “It is sunny today,” “Would you like something to eat?”) in the response generation unit. The response generation unit uses template-based or generative models (e.g., Transformer-based text generation models) to generate response sentences. The generated response sentences are passed to the speech synthesis unit, which uses neural speech synthesis models such as WaveNet or Tacotron to convert text (e.g., “It is sunny today”) into speech waveforms (e.g., 16 kHz, PCM data). Examples of output from the speech synthesis unit include a gentle female voice saying “It is sunny today,” or a calm male voice saying “Would you like something to eat?” The health monitoring unit collects biosignals obtained from heart rate sensors or body temperature sensors (e.g., heart rate data every minute, body temperature data of 36.5° C.) as time-series vectors, and detects values outside the normal range (e.g., heart rate exceeding 120 bpm, body temperature exceeding 38.0° C.) using anomaly detection algorithms (e.g., threshold judgment, time-series anomaly detection models). If an abnormality is detected, the notification unit automatically sends notification messages (e.g., “The heart rate of the elderly person has exceeded 120 bpm,” “The body temperature is 38.2° C.”) to family members or medical institutions via SMS or the Internet. These processes are realized by a series of unconventional computer processing flows, including automatic collection of sensor data, AI-based anomaly judgment, and automatic notification, which differ from conventional manual monitoring or telephone contact by humans, resulting in technical effects such as reduction of false detections, minimization of notification delays, and centralized management of data. Furthermore, the dialogue history management unit accumulates past conversation content and health condition data as a time-series database, which can be used to generate individually optimized dialogues and health advice. The emotion estimation unit estimates the emotional state of the elderly person from speech features (e.g., F0, energy, speech rate) and text content, and dynamically adjusts the tone, speed, and content of responses and speech synthesis. As a result, not only automatic responses but also personalized dialogue, health management, and emergency response tailored to the individual state and emotions of each elderly person become possible, greatly enhancing the technical value of the AI robot system. Application fields include monitoring of elderly persons living alone, remote medical support, automatic dialogue and health management in nursing care facilities, and support for persons with disabilities.

The monitoring unit can periodically measure the heart rate or body temperature of the elderly person. For example, the monitoring unit can measure the heart rate of the elderly person every hour. The monitoring unit can also measure the body temperature of the elderly person every day. Furthermore, the monitoring unit can measure the heart rate or body temperature during specific events. For example, the monitoring unit can measure the heart rate after the elderly person has exercised. As a result, the health condition of the elderly person can be monitored periodically. Specifically, the monitoring unit acquires analog signals from vital sensors such as heart rate sensors and body temperature sensors, converts them to digital signals via an A/D converter, and stores them as one-dimensional time-series vectors of heart rate data every minute (e.g., integer values from 60 to 120 bpm) and body temperature data (e.g., floating-point values from 36.0 to 38.5° C.). The monitoring unit accumulates these time-series data in local memory or a cloud database, making it possible to refer to the history for the past 24 hours or one week. The monitoring unit can perform not only periodic measurements (e.g., acquiring heart rate at 00 minutes every hour, acquiring body temperature at 7 a.m. every morning) but also additional measurements of heart rate or body temperature immediately after exercise, triggered by motion event detection signals from acceleration sensors or gyroscope sensors (e.g., start of walking, end of exercise). Furthermore, the monitoring unit applies preprocessing such as moving average or median filtering to the measured values to remove noise and correct outliers. By combining AI anomaly detection algorithms (e.g., LSTM-based time-series anomaly detection models, threshold judgment logic), values outside the normal range (e.g., heart rate exceeding 120 bpm, body temperature exceeding 38.0° C.) can be automatically detected. Inputs to the AI include one-minute heart rate vectors (e.g., [72, 74, 76, 120, 122]), body temperature vectors (e.g., [36.5, 36.7, 38.2]), and outputs include anomaly judgment labels (e.g., “normal,” “abnormal”) and anomaly scores (e.g., 0.95). The anomaly judgment results are passed to the subsequent notification unit and used as triggers for automatic notification or warning display. These series of processes are realized by unconventional computer processing, including automatic collection of sensor data, real-time analysis by AI, and flexible measurement timing control triggered by events, which differ from manual measurement and recording by humans, resulting in technical effects such as reduction of missed measurements and recording errors, faster anomaly detection, and centralized management of health data. Application fields include monitoring of elderly persons living alone, remote health management, automatic vital recording in nursing care facilities, and home medical support.

The notification unit can notify a medical institution or family when an abnormality is detected. For example, the notification unit can notify a medical institution when the heart rate of the elderly person shows an abnormal value. The notification unit can also notify the family when the body temperature of the elderly person shows an abnormal value. Furthermore, the notification unit can automatically perform notification when an abnormality is detected. For example, the notification unit can notify a medical institution when the heart rate rises sharply. As a result, prompt response is possible when an abnormality is detected. Specifically, the notification unit receives anomaly judgment labels (e.g., “heart rate abnormality,” “body temperature abnormality”) and anomaly scores (e.g., 0.98) from the monitoring unit as input, and automatically selects the notification destination (e.g., medical institution, family, care staff) and notification method (e.g., SMS, Internet API, voice call) based on a notification judgment algorithm (e.g., immediate notification when threshold is exceeded, notification when anomaly score is 0.9 or higher). The notification unit generates structured data as notification content, including the time of anomaly occurrence, measured values (e.g., “heart rate: 125 bpm,” “body temperature: 38.4° C.”), trend graphs for the past 24 hours, and reasons for anomaly judgment (e.g., “rapid increase,” “threshold exceeded”). By combining an AI-based anomaly degree estimation model (e.g., neural network that calculates anomaly scores), the priority and urgency of notification can be automatically determined, and branching processing such as notifying a medical institution when urgency is high and notifying the family when urgency is moderate can be performed. Examples of input to the AI include heart rate vectors for the last 10 minutes (e.g., [80, 82, 85, 120, 125]), body temperature vectors (e.g., [36.7, 36.8, 38.4]), and anomaly event flags (e.g., 1), and outputs include notification necessity labels (e.g., “notification required,” “notification not required”), notification destination labels (e.g., “medical institution,” “family”), and notification content templates (e.g., “The heart rate of the elderly person has rapidly increased to 125 bpm”). These outputs are passed to the notification message generation module and automatically sent via SMS or API. Unlike conventional telephone contact or manual recording by humans, these series of unconventional computer processing, including automatic analysis of sensor data, AI-based anomaly degree judgment, and automatic notification, provide technical effects such as minimization of notification delays, reduction of false reports, and standardization of notification content. Application fields include emergency monitoring of elderly persons at home, remote medical collaboration, automatic notification systems in nursing care facilities, and support for persons with disabilities.

The verbalization unit can verbalize the spoken content of the elderly person using speech recognition technology. For example, the verbalization unit can accurately verbalize the spoken content of the elderly person using deep learning-based speech recognition technology. The verbalization unit may also use HMM (Hidden Markov Model)-based speech recognition technology. As a result, the spoken content of the elderly person can be accurately verbalized. Specifically, the verbalization unit receives one-dimensional speech waveform data sampled at 16 kHz (e.g., length 16,000 to 32,000 samples, examples: “The weather is nice today,” “I'm hungry”) as input, performs preprocessing such as noise reduction and normalization, and converts it into acoustic feature vectors such as mel spectrograms or MFCCs (e.g., 128 dimensions×100 frames). Deep neural networks (e.g., convolutional neural networks, recurrent neural networks, Transformer-based speech recognition models) use these features as input and output phoneme sequences or character sequences (e.g., “kyou wa tenki ga ii ne”). In the case of HMM-based models, the most likely sequence is estimated using a combination of acoustic models, pronunciation dictionaries, and language models with algorithms such as Viterbi. Examples of input to the AI include one second of speech waveform such as “Hello,” or three seconds of speech waveform such as “I want to go to the hospital today,” and the output is string data (e.g., “Hello,” “I want to go to the hospital today”). The output text is passed to the natural language processing unit or dialogue management unit and used for subsequent intent extraction and response generation processing. Unlike conventional manual transcription or simple keyword detection by humans, these unconventional computer processing using deep learning models for high-precision speech recognition and automatic verbalization provide technical effects such as improved recognition accuracy, enhanced robustness in noisy environments, and real-time performance. Application fields include monitoring robots for elderly persons, remote dialogue support, automatic recording in nursing care settings, and support for persons with disabilities.

The response unit can generate a response based on the verbalized content. For example, the response unit can generate an appropriate response to the elderly person using natural language generation technology. The response unit may also use template-based response generation technology. As a result, an appropriate response can be generated for the elderly person. Specifically, the response unit receives text data (e.g., “The weather is nice today,” “I'm hungry”) from the verbalization unit as input, and first uses large language models (e.g., Transformer-based text generation models) or rule-based dialogue management algorithms to classify the intent of the utterance (e.g., “conversation about the weather,” “request for food”) and estimate emotion (e.g., “joy,” “sadness”). Examples of input to the AI include text such as “The weather is nice today,” past dialogue history vectors (e.g., the last five utterances), and emotion labels (e.g., “neutral”), and outputs include response candidate sentences (e.g., “Yes, it is sunny today,” “Would you like something to eat?”) and response priority scores (e.g., 0.92). In the case of template-based methods, response sentences are generated by embedding variables into predefined response templates (e.g., “Today is {weather}”) according to the intent classification result. In the case of generative models, input text and dialogue history are input into an encoder-decoder structure to automatically generate natural response sentences. The output response sentences are passed to the speech synthesis unit and used for voice output processing. Unlike conventional simple keyword responses or manual responses by humans, these unconventional computer processing using AI for intent extraction, emotion estimation, and context-aware response generation provide technical effects such as increased diversity, naturalness, and individual optimization of responses. Application fields include dialogue robots for elderly persons, remote conversation support, automatic response in nursing care settings, and support for persons with disabilities.

The output unit can output responses by voice. For example, the output unit can output the generated response as high-quality voice using speech synthesis technology. As a result, responses can be provided to the elderly person by voice. Specifically, the output unit receives text data (e.g., “It is sunny today,” “Would you like something to eat?”) from the response unit as input, and uses neural speech synthesis models (e.g., WaveNet, Tacotron, FastSpeech, etc.) to generate speech waveforms (e.g., 16 kHz, PCM data, length 2 to 5 seconds) from the text. Examples of input to the AI include text such as “It is sunny today,” speaker attributes (e.g., female, gentle voice), and emotion labels (e.g., “joy”), and outputs include speech waveform data (e.g., byte sequence), speech features (e.g., F0, speech rate, energy), etc. The speech synthesis model converts text into phoneme sequences or acoustic features with an encoder and generates waveforms with a decoder. The output unit outputs the generated speech waveform to a speaker via DAC and can dynamically adjust the volume and tone according to the hearing ability and emotions of the elderly person. Unlike conventional playback of recorded voice or simple TTS, these unconventional computer processing using deep learning models for high-quality, natural speech synthesis and personalized voice output provide technical effects such as improved intelligibility, emotional expressiveness, and real-time performance. Application fields include dialogue robots for elderly persons, remote conversation support, automatic voice guidance in nursing care settings, and support for persons with disabilities.

The provision unit can provide voice to the elderly person. For example, the provision unit can provide the output voice to the elderly person at a volume that is easy to hear. As a result, voice can be provided to the elderly person. Specifically, the provision unit receives speech waveform data (e.g., 16 kHz, PCM data, length 2 to 5 seconds) from the output unit as input, applies volume adjustment algorithms (e.g., automatic gain control, normalization) and equalizer processing, and adjusts the optimal volume and frequency characteristics according to the hearing characteristics of the elderly person and the ambient noise level (e.g., measured noise value). Examples of input to the AI include speech waveform, hearing profile (e.g., high-frequency attenuation), ambient noise level (e.g., 60 dB), and outputs include adjusted speech waveform, recommended volume value (e.g., 80 dB SPL), etc. The provision unit provides the adjusted voice to the elderly person via a speaker or bone conduction device, and can dynamically change the tone or speech rate as needed. Unlike conventional fixed-volume playback or manual adjustment by humans, these unconventional computer processing using AI for hearing and environment-adaptive voice provision provide technical effects such as improved intelligibility, comfort, and individual optimization. Application fields include dialogue robots for elderly persons, remote voice guidance, automatic voice provision in nursing care settings, and support for persons with disabilities.

The monitoring unit can monitor the health condition. For example, the monitoring unit can periodically measure the heart rate and body temperature of the elderly person and monitor the health condition. As a result, the health condition of the elderly person can always be monitored. Specifically, the monitoring unit acquires analog signals from vital sensors such as heart rate sensors and body temperature sensors, digitizes them via an A/D conversion circuit, and stores them as one-dimensional time-series vectors of heart rate data every minute (e.g., integer values from 60 to 120 bpm) and body temperature data (e.g., floating-point values from 36.0 to 38.5° C.). The monitoring unit accumulates these time-series data in local memory or a cloud database, making it possible to refer to the history for the past 24 hours or one week. The monitoring unit can perform not only periodic measurements (e.g., acquiring heart rate at 00 minutes every hour, acquiring body temperature at 7 a.m. every morning) but also additional measurements of heart rate or body temperature immediately after exercise, triggered by motion event detection signals from acceleration sensors or gyroscope sensors (e.g., start of walking, end of exercise). Furthermore, the monitoring unit applies preprocessing such as moving average or median filtering to the measured values to remove noise and correct outliers. By combining AI anomaly detection algorithms (e.g., LSTM-based time-series anomaly detection models, threshold judgment logic), values outside the normal range (e.g., heart rate exceeding 120 bpm, body temperature exceeding 38.0° C.) can be automatically detected. Inputs to the AI include one-minute heart rate vectors (e.g., [72, 74, 76, 120, 122]), body temperature vectors (e.g., [36.5, 36.7, 38.2]), and outputs include anomaly judgment labels (e.g., “normal,” “abnormal”) and anomaly scores (e.g., 0.95). The anomaly judgment results are passed to the subsequent notification unit and used as triggers for automatic notification or warning display. These series of processes are realized by unconventional computer processing, including automatic collection of sensor data, real-time analysis by AI, and flexible measurement timing control triggered by events, which differ from manual measurement and recording by humans, resulting in technical effects such as reduction of missed measurements and recording errors, faster anomaly detection, and centralized management of health data. Application fields include monitoring of elderly persons living alone, remote health management, automatic vital recording in nursing care facilities, and home medical support.

The notification unit can notify a medical institution or family in case of emergency. For example, the notification unit can automatically notify a medical institution or family when an abnormality is detected. As a result, prompt response is possible in case of emergency. Specifically, the notification unit receives anomaly judgment labels (e.g., “heart rate abnormality,” “body temperature abnormality”) and anomaly scores (e.g., 0.98) from the monitoring unit as input, and automatically selects the notification destination (e.g., medical institution, family, care staff) and notification method (e.g., SMS, Internet API, voice call) based on a notification judgment algorithm (e.g., immediate notification when threshold is exceeded, notification when anomaly score is 0.9 or higher). The notification unit generates structured data as notification content, including the time of anomaly occurrence, measured values (e.g., “heart rate: 125 bpm,” “body temperature: 38.4° C.”), trend graphs for the past 24 hours, and reasons for anomaly judgment (e.g., “rapid increase,” “threshold exceeded”). By combining an AI-based anomaly degree estimation model (e.g., neural network that calculates anomaly scores), the priority and urgency of notification can be automatically determined, and branching processing such as notifying a medical institution when urgency is high and notifying the family when urgency is moderate can be performed. Examples of input to the AI include heart rate vectors for the last 10 minutes (e.g., [80, 82, 85, 120, 125]), body temperature vectors (e.g., [36.7, 36.8, 38.4]), and anomaly event flags (e.g., 1), and outputs include notification necessity labels (e.g., “notification required,” “notification not required”), notification destination labels (e.g., “medical institution,” “family”), and notification content templates (e.g., “The heart rate of the elderly person has rapidly increased to 125 bpm”). These outputs are passed to the notification message generation module and automatically sent via SMS or API. Unlike conventional telephone contact or manual recording by humans, these series of unconventional computer processing, including automatic analysis of sensor data, AI-based anomaly degree judgment, and automatic notification, provide technical effects such as minimization of notification delays, reduction of false reports, and standardization of notification content. Application fields include emergency monitoring of elderly persons at home, remote medical collaboration, automatic notification systems in nursing care facilities, and support for persons with disabilities.

The verbalization unit can estimate emotion and adjust the verbalization expression method based on the estimated emotion. For example, if the elderly person is sad, the verbalization unit can verbalize using gentle language. If the elderly person is excited, the verbalization unit can verbalize in a calm tone. Furthermore, if the elderly person is tired, the verbalization unit can select concise and easy-to-understand words for verbalization. As a result, verbalization can be performed using an expression method adapted to the emotion of the elderly person. Emotion estimation is realized, for example, by using an emotion engine or generative AI with emotion estimation functions. Generative AI may be a text generation AI (e.g., LLM) or a multimodal generative AI, but is not limited to these examples. Specifically, the verbalization unit, in addition to speech recognition processing, extracts speech features (e.g., F0, energy, speech rate, pause length, intonation pattern) and input text content as multidimensional feature vectors (e.g., 128 dimensions×100 frames tensor, text embedding vector), and inputs them to an emotion estimation model (e.g., convolutional neural network, recurrent neural network, Transformer-based emotion classification model). The emotion estimation model, trained by supervised learning, outputs emotion labels such as “joy,” “sadness,” “anger,” “excitement,” “fatigue.” Examples of input to the AI include three seconds of speech waveform such as “I want to go to the hospital today,” its mel spectrogram, past dialogue history vectors (e.g., the last five utterances), and speaker attributes (e.g., elderly person, female). Outputs include emotion labels (e.g., “sadness”), emotion scores (e.g., 0.87), and emotion distributions (e.g., {‘joy’: 0.05, ‘sadness’: 0.87, ‘neutral’: 0.08}). The verbalization unit dynamically switches the parameters of the response generation algorithm (e.g., vocabulary selection dictionary, expression template, style conversion rules) according to the estimated emotion label. For example, in the case of a “sadness” label, gentle vocabulary and empathetic expressions are preferentially selected, and in the case of an “excitement” label, a calm tone and affirmative expressions are emphasized. Furthermore, generative AI (e.g., large language models or multimodal generative models) receives input text and emotion labels simultaneously and generates verbalized sentences optimized for emotion. Examples of input to the AI include text “I want to go to the hospital today,” emotion label “sadness,” and past conversation history vectors, and the output is a gently expressed text such as “It's okay, I will help you go to the hospital today.” The output text is passed to the subsequent response unit or speech synthesis unit and used for voice output or dialogue management. Unlike conventional simple speech recognition or manual expression adjustment by humans, these unconventional computer processing using AI for multidimensional feature analysis, emotion estimation, and expression optimization provide technical effects such as high-precision emotion-adaptive verbalization, improved naturalness and empathy in dialogue, and personalized response generation for each user. Application fields include monitoring robots for elderly persons, remote dialogue support, emotion-adaptive automatic recording in nursing care settings, support for persons with disabilities, and emotion-sensitive automatic response systems.

The verbalization unit can adjust the accuracy of verbalization according to the speaking speed or volume of the elderly person. For example, if the elderly person speaks slowly, the verbalization unit can slow down the verbalization speed. If the elderly person speaks loudly, the verbalization unit can improve the accuracy of verbalization according to the volume. Furthermore, if the elderly person speaks quickly, the verbalization unit can speed up the verbalization speed. As a result, verbalization can be performed according to the speaking speed and volume of the elderly person. Specifically, the verbalization unit extracts speech features such as speech rate (e.g., 1.5× speed, 0.8× speed), volume (e.g., RMS value, dB), utterance segment length, and pause detection from input speech waveforms (e.g., 16 kHz sampling, length 16,000 to 32,000 samples). These features are input as one-dimensional vectors (e.g., [speech rate: 1.2, volume: 65 dB, pause length: 0.3 s]) to the preprocessing unit of the speech recognition model. Examples of input to the AI include two seconds of speech such as “Good morning” (speech rate 0.9×, volume 60 dB), one second of speech such as “Thank you” (speech rate 1.5×, volume 75 dB), etc. If the speech rate is slow, the verbalization unit expands the frame length or window size of the speech recognition model and refers to more contextual information to improve recognition accuracy. Conversely, if the speech rate is fast, the verbalization unit applies shorter frame lengths or fast decoding algorithms (e.g., streaming recognition, CTC-based fast inference) to ensure real-time performance. If the volume is high, normalization or automatic gain control is applied to reduce recognition errors due to volume fluctuations. The AI output includes string data (e.g., “Good morning,” “Thank you”), recognition confidence scores (e.g., 0.98), and recognition speed (e.g., 0.5 seconds/utterance). These outputs are passed to the subsequent natural language processing unit or response generation unit. Unlike conventional speech recognition with fixed parameters or manual adjustment by humans, these unconventional computer processing using AI for dynamic parameter control and real-time optimization adapted to speech rate and volume provide technical effects such as improved recognition accuracy, enhanced robustness to various speech patterns such as fast, quiet, or loud speech, and ensured real-time performance. Application fields include monitoring robots for elderly persons, remote dialogue support, automatic recording in diverse speech environments in nursing care settings, and support for persons with disabilities.

The verbalization unit can improve the accuracy of verbalization by referring to the past conversation history of the elderly person. For example, the verbalization unit can refer to content previously spoken by the elderly person and perform more accurate verbalization on the same topic. The verbalization unit can also learn the past speech patterns of the elderly person to improve the accuracy of verbalization. Furthermore, the verbalization unit can memorize words and phrases frequently used by the elderly person and utilize them during verbalization. As a result, referring to past conversation history improves the accuracy of verbalization. Specifically, the verbalization unit cooperates with the dialogue history management unit to obtain past conversation content (e.g., utterance text for the past week, frequently used phrase list, topic-specific speech patterns) from a time-series database. Examples of input to the AI include current speech waveform (e.g., “I want to go to the hospital today”), past utterance text on the same topic (e.g., “You said you wanted to go to the hospital last week as well”), and frequently used vocabulary vectors (e.g., [‘hospital’, ‘medicine’, ‘walk’]). The verbalization unit inputs past speech patterns and frequently used vocabulary as bias information to the decoder of the speech recognition model and dynamically corrects the probability distribution of beam search or the language model. For example, if the word “hospital” frequently appears in the past, the verbalization unit prioritizes recognizing “hospital” even for acoustically ambiguous input. Furthermore, the verbalization unit learns user-specific speech tendencies (e.g., sentence-ending characteristics, abbreviation usage tendencies) and can construct individually optimized speech recognition models (e.g., user-adaptive Transformer speech recognition models). The AI output includes string data (e.g., “I want to go to the hospital today”), recognition confidence scores (e.g., 0.99), and history reference flags (e.g., history referenced). These outputs are passed to the subsequent natural language processing unit or response generation unit. Unlike conventional speech recognition without history reference or manual correction by humans, these unconventional computer processing using AI for history reference, individual optimization, and dynamic bias correction provide technical effects such as improved recognition accuracy, continuous understanding of the same topic, and personalized verbalization for each user. Application fields include monitoring robots for elderly persons, remote dialogue support, individual recording in nursing care settings, and support for persons with disabilities.

The verbalization unit can estimate the emotion of the elderly person and adjust the timing of verbalization based on the estimated emotion. For example, if the elderly person is feeling down, the verbalization unit can wait a moment before verbalizing. If the elderly person is excited, the verbalization unit can verbalize immediately. Furthermore, if the elderly person is tired, the verbalization unit can verbalize at a slower timing. As a result, verbalization can be performed at a timing adapted to the emotion of the elderly person. Emotion estimation is realized, for example, by using an emotion engine or generative AI with emotion estimation functions. Generative AI may be a text generation AI (e.g., LLM) or a multimodal generative AI, but is not limited to these examples. Specifically, the verbalization unit inputs speech features extracted from speech input (e.g., F0, speech rate, pause length, energy) and input text content to an emotion estimation model (e.g., Transformer-based emotion classification model), and outputs emotion labels (e.g., “feeling down,” “excited,” “fatigue”) and emotion scores (e.g., 0.92). Examples of input to the AI include two seconds of speech such as “I'm tired today,” mel spectrogram, and past emotion history vectors (e.g., the last five emotion estimation results). The verbalization unit dynamically controls the timing control module of the verbalization process according to the estimated emotion label. For example, in the case of a “feeling down” label, a certain delay (e.g., 1.5 seconds) is inserted before generating the verbalized sentence; in the case of an “excited” label, the verbalized sentence is generated immediately; and in the case of a “fatigue” label, verbalization is performed at a slower timing (e.g., 1.2 times the usual delay). The AI output includes verbalized sentences (e.g., “Thank you for your hard work today”), timing control signals (e.g., delay 1.5 seconds), and emotion labels (e.g., “feeling down”). These outputs are passed to the subsequent response unit or speech synthesis unit and contribute to improved naturalness of dialogue and user experience. Unlike conventional fixed-timing responses or manual timing adjustment by humans, these unconventional computer processing using AI for emotion-adaptive timing control and real-time emotion estimation provide technical effects such as improved naturalness and empathy in dialogue and personalized response timing control for each user. Application fields include monitoring robots for elderly persons, remote dialogue support, emotion-adaptive automatic recording in nursing care settings, and support for persons with disabilities.

The verbalization unit can apply region-specific speech recognition models to accommodate the dialects and accents of the elderly person. For example, if the elderly person speaks in Kansai dialect, the verbalization unit can use a speech recognition model adapted to Kansai dialect. If the elderly person speaks in Tohoku dialect, the verbalization unit can use a speech recognition model adapted to Tohoku dialect. Furthermore, if the elderly person speaks in Okinawa dialect, the verbalization unit can use a speech recognition model adapted to Okinawa dialect. As a result, verbalization can be performed in accordance with the dialects and accents of the elderly person. Specifically, the verbalization unit extracts acoustic features (e.g., mel spectrogram, MFCC, intonation pattern) from input speech waveforms and inputs them to a region determination module (e.g., dialect classification model using convolutional neural networks). Examples of input to the AI include two seconds of speech such as “ookini” (Kansai dialect), three seconds of speech such as “ndabe” (Tohoku dialect), etc. The region determination module outputs dialect labels (e.g., “Kansai dialect,” “Tohoku dialect,” “Okinawa dialect”) based on acoustic features and vocabulary distribution. The verbalization unit automatically selects and executes the corresponding speech recognition model (e.g., Transformer speech recognition model for Kansai dialect, recurrent neural network model for Tohoku dialect) according to the estimated dialect label. The AI output includes string data (e.g., “ookini,” “ndabe”), recognition confidence scores (e.g., 0.97), and dialect labels (e.g., “Kansai dialect”). Furthermore, the verbalization unit can learn user-specific speech history and regional information to construct individually optimized dialect models. These outputs are passed to the subsequent natural language processing unit or response generation unit. Unlike conventional speech recognition limited to standard Japanese or manual dialect conversion by humans, these unconventional computer processing using AI for dialect and accent-adaptive speech recognition and automatic model switching provide technical effects such as improved recognition accuracy, enhanced adaptability to diverse regional speech, and personalized verbalization for each user. Application fields include monitoring robots for elderly persons, remote dialogue support, multi-region adaptive automatic recording in nursing care settings, and support for persons with disabilities.

The verbalization unit can emphasize specific keywords in verbalization according to the spoken content of the elderly person. For example, if the elderly person says “I want to go to the hospital,” the verbalization unit can emphasize “hospital” in verbalization. If the elderly person says “I forgot to take my medicine,” the verbalization unit can also emphasize “medicine.” Furthermore, if the elderly person says “I want to go for a walk,” the verbalization unit can also emphasize “walk.” As a result, verbalization can be performed according to the spoken content of the elderly person. Specifically, the verbalization unit passes the text data after speech recognition (e.g., “I want to go to the hospital”) to the natural language processing unit, and extracts important keywords (e.g., “hospital,” “medicine,” “walk”) using keyword extraction algorithms (e.g., TF-IDF, Transformer with attention mechanism, rule-based dictionary). Examples of input to the AI include text “I forgot to take my medicine,” past conversation history vectors, and important keyword dictionaries. The verbalization unit assigns emphasis tags to the extracted keywords (e.g., <em>hospital</em>), attaches emphasis instructions for speech synthesis (e.g., pitch increase, volume increase, speech rate decrease), and adds metadata. The AI output includes text data with emphasis (e.g., “<em>hospital</em>ni ikitai”), emphasis instruction vectors (e.g., [hospital: emphasis level 0.8]), etc. These outputs are passed to the subsequent speech synthesis unit or response generation unit and reflected as emphasis expressions during voice output. Unlike conventional simple text conversion or manual emphasis by humans, these unconventional computer processing using AI for keyword extraction, emphasis instruction assignment, and speech synthesis linkage provide technical effects such as improved transmission of important information, emphasis of urgency and importance, and enhanced user experience. Application fields include monitoring robots for elderly persons, remote dialogue support, automatic emphasis of important matters in nursing care settings, and support for persons with disabilities.

The response unit can estimate the emotion of the elderly person and adjust the tone and content of the response based on the estimated emotion. For example, if the elderly person is sad, the response unit can respond with encouraging words in a gentle tone. If the elderly person is excited, the response unit can respond in a calm tone. Furthermore, if the elderly person is tired, the response unit can respond with concise and easy-to-understand content. As a result, responses can be provided with a tone and content adapted to the emotion of the elderly person. Emotion estimation is realized, for example, by using an emotion engine or generative AI with emotion estimation functions. Generative AI may be a text generation AI (e.g., LLM) or a multimodal generative AI, but is not limited to these examples. Specifically, the response unit receives text data (e.g., “I want to go to the hospital today,” “I haven't been feeling well lately”) from the verbalization unit, speech features (e.g., F0, speech rate, energy), and past dialogue history vectors (e.g., the last five utterances and emotion estimation results) as multidimensional input tensors to an emotion estimation model (e.g., Transformer-based emotion classification model, convolutional neural network, recurrent neural network). The emotion estimation model, trained by supervised learning, outputs emotion labels such as “sadness,” “excitement,” “fatigue,” “joy.” Examples of input to the AI include text “I want to go to the hospital today,” speech feature vectors (e.g., F0=180 Hz, speech rate=0.8×, energy=low), and past emotion history (e.g., previous “sadness”). AI outputs include emotion labels (e.g., “sadness”), emotion scores (e.g., 0.87), and emotion distributions (e.g., sadness 0.87, joy 0.05, neutral 0.08). The response unit dynamically switches the parameters of the response generation algorithm (e.g., vocabulary selection dictionary, expression template, style conversion rules, tone control parameters) according to the estimated emotion label. For example, in the case of a “sadness” label, gentle vocabulary and empathetic expressions are preferentially selected, and in the case of an “excitement” label, a calm tone and affirmative expressions are emphasized. Response generation may use template-based methods (e.g., “It's okay, please rest slowly”) or generative models (e.g., context-optimized response generation by large language models). Examples of input to the AI include text “I haven't been feeling well lately,” emotion label “sadness,” and past conversation history vectors, and the output is a gently expressed text such as “Don't overdo it, please let me know if there is anything I can help you with.” The output response sentences are passed to the speech synthesis unit and used for voice output processing. Unlike conventional simple keyword responses or manual responses by humans, these unconventional computer processing using AI for multidimensional feature analysis, emotion estimation, and expression optimization provide technical effects such as increased diversity, naturalness, empathy, and individual optimization of responses. Application fields include dialogue robots for elderly persons, remote conversation support, emotion-adaptive automatic response in nursing care settings, support for persons with disabilities, and emotion-sensitive automatic dialogue systems.

The response unit can generate a more personalized response by referring to the past conversation history of the elderly person. For example, the response unit can refer to content previously spoken by the elderly person and respond to the same topic. The response unit can also learn the past speech patterns of the elderly person to generate personalized responses. Furthermore, the response unit can memorize words and phrases frequently used by the elderly person and utilize them during response generation. As a result, referring to past conversation history enables personalized responses. Specifically, the response unit cooperates with the dialogue history management unit to obtain past conversation content (e.g., utterance text for the past week, frequently used phrase list, topic-specific speech patterns) from a time-series database. Examples of input to the AI include current utterance text (e.g., “I want to go to the hospital today”), past utterance text on the same topic (e.g., “You said you wanted to go to the hospital last week as well”), frequently used vocabulary vectors (e.g., [‘hospital’, ‘medicine’, ‘walk’]), and past response history (e.g., previous “When going to the hospital, be careful not to forget anything”). The response unit inputs these history information as bias information to the natural language processing model (e.g., large language model, rule-based dialogue management algorithm) and preferentially reflects past speech tendencies and frequently used vocabulary during response generation. For example, if the word “hospital” frequently appears in the past, the response unit automatically includes related advice or cautions in the response. Furthermore, the response unit learns user-specific speech tendencies (e.g., sentence-ending characteristics, abbreviation usage tendencies) and can construct individually optimized response generation models (e.g., user-adaptive Transformer text generation models). AI outputs include response candidate sentences (e.g., “You have plans to go to the hospital today, do you have everything you need?”), response priority scores (e.g., 0.95), and history reference flags (e.g., history referenced). These outputs are passed to the speech synthesis unit and used for voice output processing. Unlike conventional responses without history reference or manual correction by humans, these unconventional computer processing using AI for history reference, individual optimization, and dynamic bias correction provide technical effects such as personalized optimization of responses, continuous understanding of the same topic, and personalized dialogue for each user. Application fields include monitoring robots for elderly persons, remote dialogue support, individual recording and response in nursing care settings, and support for persons with disabilities.

The response unit can generate a response including appropriate advice or cautions according to the health condition of the elderly person. For example, if the heart rate of the elderly person is high, the response unit can advise them to calm down. If the body temperature of the elderly person is high, the response unit can respond by encouraging hydration. Furthermore, if the health condition of the elderly person is not good, the response unit can respond by recommending a visit to a medical institution. As a result, responses can be provided according to the health condition of the elderly person. Specifically, the response unit receives the latest biological information (e.g., heart rate vector [80, 85, 120], body temperature vector [36.7, 38.2], anomaly judgment label “heart rate abnormality”) and past health history data from the health monitoring unit as input. Examples of input to the AI include heart rate vectors for the last 10 minutes, body temperature vectors, anomaly event flags (e.g., 1), and health condition labels (e.g., “high body temperature”). The response unit uses health condition judgment algorithms (e.g., threshold judgment, LSTM-based time-series anomaly detection models) and large language models to automatically generate response templates according to the health condition (e.g., “Your heart rate is high, so please take a rest,” “Your body temperature is high, so I recommend hydration,” “If you are not feeling well, please visit a medical institution”). AI outputs include response sentences (e.g., “Your heart rate seems high, so please take a break without overexerting yourself”), advice type labels (e.g., “caution,” “recommendation to visit”), and response priority scores (e.g., 0.98). These outputs are passed to the speech synthesis unit and used for voice output processing. Unlike conventional manual health advice or simple fixed responses by humans, these unconventional computer processing using AI for health data analysis, anomaly detection, and individually optimized advice generation provide technical effects such as rapid and accurate response generation according to health condition, reduction of false detections, and automation of health management. Application fields include monitoring robots for elderly persons, remote health management, automatic health advice in nursing care settings, and support for persons with disabilities.

The response unit can estimate the emotion of the elderly person and adjust the length of the response based on the estimated emotion. For example, when the elderly person is feeling down, the response unit can generate a longer response with encouraging words. When the elderly person is excited, the response unit can generate a shorter response to help calm them down. Furthermore, when the elderly person is tired, the response unit can provide a concise response. This enables responses to be given with a length appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the response unit inputs text data received from the verbalization unit, voice feature quantities (e.g., F0, speech rate, energy), and past emotion history vectors into an emotion estimation model (e.g., Transformer-based emotion classification model), and outputs emotion labels (e.g., “depression,” “excitement,” “fatigue”) and emotion scores (e.g., 0.92). Examples of input to the AI include text such as “I'm tired today,” voice feature vectors, and past emotion history (e.g., results of the last five emotion estimations). The response unit dynamically adjusts parameters of the response generation algorithm (e.g., response sentence length, level of detail, vocabulary selection) according to the estimated emotion label. For example, for a “depression” label, a longer response (e.g., “You seem tired lately. Please take it easy and rest. Let me know if there is anything I can help you with.”) is generated; for an “excitement” label, a short and calm response (e.g., “It's okay, let's take it slow.”) is generated; and for a “fatigue” label, a concise response (e.g., “Thank you for your hard work.”) is generated. The AI output includes the response sentence, response length label (e.g., “long sentence,” “short sentence,” “concise”), emotion label, and so on. These outputs are passed to the speech synthesis unit and used for voice output processing. Unlike conventional fixed-length responses or manual adjustment by humans, this non-conventional computer processing by AI—emotion-adaptive response length control and real-time emotion estimation—brings technical effects such as improved naturalness and empathy in dialogue, and personalized response length control for each user. Application fields include elderly monitoring robots, remote dialogue support, emotion-adaptive automatic response in care settings, and support for people with disabilities.

The response unit can generate responses that include relevant information based on the hobbies and interests of the elderly person. For example, if the elderly person is interested in gardening, the response unit can include information about seasonal flowers in the response. If the elderly person is interested in cooking, the response unit can include a simple recipe in the response. Furthermore, if the elderly person is interested in travel, the response unit can include information about travel destinations in the response. This enables responses tailored to the hobbies and interests of the elderly person. Specifically, the response unit collaborates with the dialogue history management unit and user profile management unit to obtain the elderly person's hobby and interest database (e.g., hobby labels such as “gardening,” “cooking,” “travel,” interest score, and past spoken content). Examples of input to the AI include current utterance text (e.g., “I've been enjoying gardening lately”), hobby label (e.g., “gardening”), interest score (e.g., 0.95), and past hobby-related utterance history. The response unit inputs this information into a natural language processing model (e.g., large language model, rule-based dialogue management algorithm), extracts appropriate information from knowledge bases related to hobbies and interests (e.g., seasonal flower lists, simple recipe collections, travel destination information databases), and incorporates it into the response sentence. AI output includes response sentences (e.g., “Tulips are blooming beautifully this season,” “Today, I will introduce a simple vegetable soup recipe,” “Kyoto is recommended as your next travel destination”), related information labels (e.g., “gardening information,” “recipe information”), and response priority scores. These outputs are passed to the speech synthesis unit and used for voice output processing. Unlike conventional responses that do not consider hobbies or manual information provision by humans, this non-conventional computer processing by AI—hobby and interest-adaptive information extraction and automatic response generation—brings technical effects such as improved user experience, activation of hobby activities, and individually optimized information provision. Application fields include elderly monitoring robots, remote dialogue support, hobby activity support in care settings, and support for people with disabilities.

The response unit can adjust the order and priority of responses according to the content spoken by the elderly person. For example, if the elderly person brings up an urgent topic, the response unit can respond to it with the highest priority. If the elderly person brings up multiple topics, the response unit can determine the order of responses according to their importance. Furthermore, if the elderly person brings up everyday topics, the response unit can respond in a relaxed order. This enables responses to be given in an order and priority appropriate to the content spoken by the elderly person. Specifically, the response unit receives text data from the verbalization unit (e.g., “I want to go to the hospital today, and I forgot to take my medicine”), topic classification labels (e.g., “urgent,” “health,” “daily”), and past dialogue history vectors as input. Examples of input to the AI include text containing multiple topics (e.g., “I want to go to the hospital today, and I forgot to take my medicine”), topic classification labels (e.g., “urgent,” “health”), and topic importance scores (e.g., [0.98, 0.85]). The response unit uses topic classification algorithms (e.g., Transformer-based topic classification models, rule-based classifiers) and priority determination logic (e.g., urgency score calculation, importance ranking) to determine the priority of each topic and add order information to the response generation algorithm. AI output includes a list of response sentences (e.g., first: “You have a plan to go to the hospital, do you have everything you need?”; second: “Please make sure not to forget to take your medicine”), response order labels (e.g., “urgent priority”), and priority scores for each response. These outputs are passed to the speech synthesis unit and played in order during voice output. Unlike conventional responses that do not consider order or manual order adjustment by humans, this non-conventional computer processing by AI—topic classification, priority determination, and order optimization—brings technical effects such as rapid response to urgent topics, efficient handling of multiple topics, and improved user experience. Application fields include elderly monitoring robots, remote dialogue support, automatic multi-topic response in care settings, and support for people with disabilities.

The output unit can estimate the emotion of the elderly person and adjust the tone and speed of the output voice based on the estimated emotion. For example, when the elderly person is sad, the output unit can output the voice in a gentle tone and at a slow speed. When the elderly person is excited, the output unit can output the voice in a calm tone. Furthermore, when the elderly person is tired, the output unit can output the voice in a concise and easy-to-understand tone. This enables the output of voice with a tone and speed appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the output unit inputs text data received from the response unit (e.g., “I want to go to the hospital today,” “I haven't been feeling well lately”), voice feature quantities (e.g., F0, speech rate, energy), and past emotion estimation history vectors (e.g., the last five emotion labels and scores) as a multidimensional tensor into an emotion estimation model (e.g., Transformer-based emotion classification model, convolutional neural network, recurrent neural network). This emotion estimation model, trained by supervised learning, outputs emotion labels such as “sadness,” “excitement,” “fatigue,” “joy,” etc. Examples of input to the AI include text such as “I want to go to the hospital today,” voice feature vectors (e.g., F0=180 Hz, speech rate=0.8×, energy=low), and past emotion history (e.g., previous “sadness”). AI output includes emotion labels (e.g., “sadness”), emotion scores (e.g., 0.87), and emotion distribution (e.g., sadness 0.87, joy 0.05, neutral 0.08). The output unit dynamically switches parameters of the speech synthesis model (e.g., WaveNet, Tacotron, FastSpeech, etc.), such as tone control parameters, speech rate control parameters, and prosody control vectors, according to the estimated emotion label. For example, for a “sadness” label, voice is generated with a gentle tone (e.g., lower F0, speech rate at 0.8×, suppressed energy); for an “excitement” label, voice is generated with a calm tone (e.g., stabilized F0, normal speech rate, moderate energy); and for a “fatigue” label, voice is generated with a concise and clear tone (e.g., slightly slower speech rate, increased clarity). Examples of AI output include voice waveform data (e.g., 16 kHz, PCM data, length 2-5 seconds), voice feature quantities (e.g., F0=160 Hz, speech rate=0.8×, energy=low), and metadata with emotion labels. These outputs are provided to the elderly person via DAC and speakers as voice optimized for their emotional state. Unlike conventional fixed-tone and fixed-speed voice output or manual adjustment by humans, this non-conventional computer processing by AI—emotion-adaptive tone and speed control and real-time optimization—brings technical effects such as improved audibility, empathy, and personalized voice output. Application fields include elderly dialogue robots, remote conversation support, emotion-adaptive automatic voice guidance in care settings, support for people with disabilities, and emotion-sensitive automatic response systems.

The output unit can adjust the volume and frequency of the output voice according to the hearing ability of the elderly person. For example, when the elderly person's hearing is impaired, the output unit can increase the volume of the voice. When the elderly person's hearing is good, the output unit can output the voice at an appropriate volume. Furthermore, the output unit can adjust the frequency of the voice according to the hearing ability of the elderly person. This enables the output of voice with a volume and frequency appropriate to the hearing ability of the elderly person. Specifically, the output unit receives the hearing profile (e.g., high-frequency attenuation, low-frequency sensitivity, hearing level in dB SPL) obtained from the user profile management unit and the environmental sound level (e.g., noise measurement value of 60 dB) as input. Examples of input to the AI include voice waveform data (e.g., 16 kHz, PCM data), hearing profile vector (e.g., [high-frequency −10 dB, mid-frequency −5 dB, low-frequency 0 dB]), and environmental sound level (e.g., 65 dB). The output unit applies volume adjustment algorithms (e.g., automatic gain control, normalization), equalizer processing (e.g., high-frequency boost, low-frequency cut), and AI-based hearing-adaptive voice optimization models (e.g., neural network-based frequency characteristic optimization) to generate voice waveforms optimized for the individual hearing characteristics of the elderly person. AI output includes adjusted voice waveform data (e.g., 16 kHz, PCM data, volume 80 dB SPL, high-frequency +6 dB correction), recommended volume value (e.g., 80 dB SPL), and frequency characteristic correction parameters (e.g., high-frequency +6 dB, low-frequency −2 dB). These outputs are provided to the elderly person via speakers or bone conduction devices, and the tone and speech rate of the voice may also be dynamically changed as needed. Unlike conventional fixed-volume and fixed-frequency playback or manual adjustment by humans, this non-conventional computer processing by AI—hearing and environment-adaptive voice optimization and real-time control—brings technical effects such as improved audibility, comfort, individual optimization, and enhanced accessibility for people with hearing loss. Application fields include elderly dialogue robots, remote voice guidance, automatic voice provision in care settings, support for people with disabilities, and hearing compensation-type automatic response systems.

The output unit can optimize the content of the output voice by referring to the past reactions of the elderly person. For example, the output unit can refer to the voice tone that the elderly person preferred in the past and output the voice in the same tone. The output unit can also analyze the past reactions of the elderly person and output the voice with optimal content. Furthermore, the output unit can remember the content to which the elderly person responded in the past and reflect it in the output voice. This enables the output of voice with optimal content by referring to past reactions. Specifically, the output unit collaborates with the dialogue history management unit and user profile management unit to obtain past voice output history (e.g., tone, speech rate, content, user reaction labels such as “positive reaction,” “no reaction,” “discomfort” over the past week) from a time-series database. Examples of input to the AI include current output candidate text (e.g., “I want to go to the hospital today”), past preferred tone (e.g., gentle voice, speech rate 0.9×), and past reaction history vector (e.g., [‘positive reaction’, ‘positive reaction’, ‘no reaction’]). The output unit uses an AI model that analyzes past reaction patterns (e.g., recurrent neural network, reinforcement learning-based optimization model) to estimate optimal tone, speech rate, and content selection parameters. For example, if a “gentle tone” for “I want to go to the hospital” received a positive reaction in the past, the voice is generated with a similar tone and speech rate. AI output includes optimized voice waveform data (e.g., 16 kHz, PCM data, gentle voice, speech rate 0.9×), tone selection label (e.g., “gentle”), and content optimization flag (e.g., history referenced). These outputs are provided to the elderly person via speakers and contribute to improved user experience. Unlike conventional history-non-referenced voice output or manual optimization by humans, this non-conventional computer processing by AI—history reference, individual optimization, and reinforcement learning-based optimization—brings technical effects such as personalized voice output, improved user satisfaction, and continuous optimization. Application fields include elderly dialogue robots, remote conversation support, individually optimized voice guidance in care settings, and support for people with disabilities.

The output unit can estimate the emotion of the elderly person and adjust the timing of the output voice based on the estimated emotion. For example, when the elderly person is feeling down, the output unit can output the voice after a short pause. When the elderly person is excited, the output unit can output the voice immediately. Furthermore, when the elderly person is tired, the output unit can output the voice at a slower timing. This enables the output of voice with timing appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the output unit inputs text data received from the response unit or verbalization unit, voice feature quantities (e.g., F0, speech rate, energy), and past emotion estimation history vectors into an emotion estimation model (e.g., Transformer-based emotion classification model), and outputs emotion labels (e.g., “depression,” “excitement,” “fatigue”) and emotion scores (e.g., 0.92). Examples of input to the AI include a two-second utterance of “I'm tired today,” mel spectrogram, and past emotion history vectors (e.g., results of the last five emotion estimations). The output unit dynamically controls the voice output timing control module according to the estimated emotion label. For example, for a “depression” label, a certain delay (e.g., 1.5 seconds) is inserted before outputting the voice; for an “excitement” label, the voice is output immediately; and for a “fatigue” label, the voice is output at a slower timing (e.g., 1.2 times the normal delay). AI output includes voice waveform data, timing control signals (e.g., 1.5-second delay), and emotion labels. These outputs are provided to the elderly person via speakers and contribute to improved naturalness of dialogue and user experience. Unlike conventional fixed-timing voice output or manual timing adjustment by humans, this non-conventional computer processing by AI—emotion-adaptive timing control and real-time emotion estimation—brings technical effects such as improved naturalness and empathy in dialogue, and personalized voice output timing control for each user. Application fields include elderly monitoring robots, remote dialogue support, emotion-adaptive automatic voice guidance in care settings, and support for people with disabilities.

The output unit can detect environmental sounds of the elderly person and automatically adjust the volume of the output voice. For example, when the surroundings of the elderly person are noisy, the output unit can increase the volume of the voice. When the surroundings are quiet, the output unit can output the voice at an appropriate volume. Furthermore, the output unit can automatically adjust the volume of the voice according to the environmental sounds. This enables the output of voice with a volume appropriate to the environmental sounds. Specifically, the output unit obtains environmental sound level data (e.g., in dB SPL, 60-90 dB) in real time from environmental sound sensors (e.g., microphones, noise meters), and applies volume adjustment algorithms (e.g., automatic gain control, normalization) and AI-based environment-adaptive volume optimization models (e.g., neural network-based volume recommendation estimation). Examples of input to the AI include environmental sound level (e.g., 75 dB), past volume adjustment history, and user hearing profile. When the environmental sound is noisy, the output unit automatically increases the volume (e.g., +10 dB correction); when it is quiet, the volume is adjusted to an appropriate level (e.g., 60 dB). AI output includes adjusted voice waveform data (e.g., 16 kHz, PCM data, volume 80 dB SPL), recommended volume value (e.g., 80 dB SPL), and environmental sound labels (e.g., “noise,” “silence”). These outputs are provided to the elderly person via speakers and contribute to improved audibility. Unlike conventional fixed-volume playback or manual volume adjustment by humans, this non-conventional computer processing by AI—environmental sound-adaptive volume control and real-time optimization—brings technical effects such as improved audibility, comfort, and automation. Application fields include elderly dialogue robots, remote voice guidance, automatic volume adjustment in care settings, and support for people with disabilities.

The output unit can adjust the emphasized portions of the output voice according to the content spoken by the elderly person. For example, when the elderly person brings up an important topic, the output unit can emphasize the voice output. When the elderly person brings up everyday topics, the output unit can output the voice in a relaxed tone. Furthermore, when the elderly person brings up an urgent topic, the output unit can quickly emphasize the voice output. This enables the output of voice with emphasized portions appropriate to the content spoken. Specifically, the output unit passes text data received from the response unit or verbalization unit (e.g., “I want to go to the hospital,” “I forgot to take my medicine”) to the natural language processing unit, and uses keyword extraction algorithms (e.g., TF-IDF, Transformer with attention mechanism, rule-based dictionary) to extract important words (e.g., “hospital,” “medicine,” “urgent”). Examples of input to the AI include text such as “I forgot to take my medicine,” past conversation history vectors, and important word dictionaries. The output unit assigns emphasis tags (e.g., pitch increase, volume increase, speech rate decrease during speech synthesis) as metadata to the extracted keywords and inputs emphasis instruction vectors to the speech synthesis model (e.g., WaveNet, Tacotron, etc.). AI output includes voice waveform data with emphasis (e.g., volume +6 dB and pitch +20 Hz only for keyword portions), emphasis instruction vectors (e.g., [hospital: emphasis level 0.8]), and emphasis labels (e.g., “urgent”). These outputs are provided to the elderly person via speakers and contribute to improved transmission of important information and emphasis of urgency. Unlike simple text conversion or manual emphasis by humans, this non-conventional computer processing by AI—keyword extraction, emphasis instruction assignment, and speech synthesis integration—brings technical effects such as improved transmission of important information, emphasis of urgency and importance, and improved user experience. Application fields include elderly monitoring robots, remote dialogue support, automatic emphasis of important matters in care settings, and support for people with disabilities.

The provision unit can estimate the emotion of the elderly person and adjust the content of the provided voice based on the estimated emotion. For example, when the elderly person is sad, the provision unit can provide encouraging words. When the elderly person is excited, the provision unit can provide words to calm them down. Furthermore, when the elderly person is tired, the provision unit can provide words to help them relax. This enables the provision of voice with content appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the provision unit inputs the elderly person's utterance text received from the speech recognition unit or response unit (e.g., “I haven't been feeling well lately,” “I'm tired today”), voice feature quantities (e.g., F0, speech rate, energy), and past emotion estimation history vectors (e.g., the last five emotion labels and scores) as a multidimensional tensor into an emotion estimation model (e.g., Transformer-based emotion classification model, convolutional neural network, recurrent neural network). This emotion estimation model, trained by supervised learning, outputs emotion labels such as “sadness,” “excitement,” “fatigue,” “joy,” etc. Examples of input to the AI include text such as “I'm tired today,” voice feature vectors (e.g., F0=170 Hz, speech rate=0.7×, energy=low), and past emotion history (e.g., previous “fatigue”). AI output includes emotion labels (e.g., “sadness”), emotion scores (e.g., 0.91), and emotion distribution (e.g., sadness 0.91, joy 0.04, neutral 0.05). The provision unit dynamically switches vocabulary selection dictionaries, expression templates, and style conversion rules for the voice content generation algorithm (e.g., large language model, template-based generation, rule-based expression optimization) according to the estimated emotion label. For example, for a “sadness” label, gentle vocabulary and empathetic expressions (e.g., “Please don't overdo it, let me know if there is anything I can help you with”) are preferentially selected; for an “excitement” label, calm tone and affirmative expressions (e.g., “Let's take a deep breath slowly”) are emphasized; and for a “fatigue” label, expressions that encourage relaxation (e.g., “Please take it easy and rest today”) are generated. Examples of input to the AI include text such as “I haven't been feeling well lately,” emotion label “sadness,” and past conversation history vectors, and the output is gentle expression text such as “Please don't overdo it, let me know if there is anything I can help you with.” The generated voice content is passed to the speech synthesis unit and generated as a voice waveform. Furthermore, the provision unit can learn the reaction history and preferences of each user and apply individually optimized expression patterns. This series of processing, unlike conventional fixed expressions or manual adjustment by humans, is non-conventional computer processing by AI—multidimensional feature analysis, emotion estimation, and expression optimization—bringing technical effects such as high-precision emotion-adaptive voice content generation, improved naturalness and empathy in dialogue, and personalized response generation for each user. Application fields include elderly monitoring robots, remote dialogue support, emotion-adaptive automatic voice guidance in care settings, support for people with disabilities, and emotion-sensitive automatic response systems.

The provision unit can optimize the timing of the provided voice by referring to the past reactions of the elderly person. For example, the provision unit can provide voice at the timing preferred by the elderly person in the past. The provision unit can also analyze the past reactions of the elderly person and provide voice at the optimal timing. Furthermore, the provision unit can remember the timing at which the elderly person responded in the past and reflect it in the voice provision. This enables the provision of voice at the optimal timing by referring to past reactions. Specifically, the provision unit collaborates with the dialogue history management unit and user profile management unit to obtain past voice provision history (e.g., voice output timing, content, user reaction labels such as “positive reaction,” “no reaction,” “discomfort” over the past week) from a time-series database. Examples of input to the AI include current voice output candidate text (e.g., “I want to go to the hospital today”), past preferred timing (e.g., 8 a.m., before meals), and past reaction history vector (e.g., [‘positive reaction’, ‘positive reaction’, ‘no reaction’]). The provision unit uses an AI model that analyzes past reaction patterns (e.g., recurrent neural network, reinforcement learning-based optimization model) to estimate optimal voice output timing parameters. For example, if providing voice in a “gentle tone” before breakfast received a positive reaction in the past, the provision unit controls the timing to provide voice in a similar manner. AI output includes voice output timing control signals (e.g., output at 8 a.m., output before meals), reaction prediction scores (e.g., 0.93), and history reference flags (e.g., history referenced). These outputs are passed to the speech synthesis unit and speaker control module, and voice is provided to the elderly person at the optimal timing. Unlike conventional fixed-timing voice output or manual optimization by humans, this non-conventional computer processing by AI—history reference, individual optimization, and reinforcement learning-based timing optimization—brings technical effects such as personalized voice provision, improved user satisfaction, and continuous optimization. Application fields include elderly dialogue robots, remote conversation support, individually optimized voice guidance in care settings, and support for people with disabilities.

The provision unit can customize the content of the provided voice according to the health condition of the elderly person. For example, when the heart rate of the elderly person is high, the provision unit can provide voice advice to help them calm down. When the body temperature of the elderly person is high, the provision unit can provide voice advice to encourage hydration. Furthermore, when the health condition of the elderly person is not good, the provision unit can provide voice advice to recommend visiting a medical institution. This enables the provision of voice with content appropriate to the health condition. Specifically, the provision unit receives the latest biometric information (e.g., heart rate vector [80, 85, 120], body temperature vector [36.7, 38.2], abnormality judgment label “heart rate abnormality”) and past health history data from the health monitoring unit. Examples of input to the AI include heart rate vector for the last 10 minutes, body temperature vector, abnormal event flag (e.g., 1), and health condition label (e.g., “high body temperature”). The provision unit uses health condition judgment algorithms (e.g., threshold judgment, LSTM-based time-series anomaly detection model) and large language models to automatically generate voice content templates according to the health condition (e.g., “Your heart rate is high, so please take a rest,” “Your body temperature is high, so we recommend hydration,” “If you are not feeling well, please visit a medical institution”). AI output includes voice content text (e.g., “Your heart rate seems high, so please take a break without overexerting yourself”), advice type label (e.g., “caution,” “recommendation to visit”), and content priority score (e.g., 0.98). These outputs are passed to the speech synthesis unit and used for voice output processing. Furthermore, the provision unit can learn the health history and reactions of each user and apply individually optimized advice expressions. Unlike manual health advice or simple template responses by humans, this non-conventional computer processing by AI—health data analysis, anomaly detection, and individually optimized advice generation—brings technical effects such as rapid and accurate voice content generation according to health condition, reduction of false detections, and automation of health management. Application fields include elderly monitoring robots, remote health management, automatic health advice in care settings, and support for people with disabilities.

The provision unit can estimate the emotion of the elderly person and adjust the order of the provided voice based on the estimated emotion. For example, when the elderly person is feeling down, the provision unit can provide encouraging words first. When the elderly person is excited, the provision unit can provide words to calm them down first. Furthermore, when the elderly person is tired, the provision unit can provide words to help them relax first. This enables the provision of voice in an order appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the provision unit receives multiple voice output candidate texts (e.g., “I want to go to the hospital today,” “I forgot to take my medicine”), emotion estimation labels (e.g., “depression,” “excitement,” “fatigue”), and past emotion history vectors from the response unit or verbalization unit. Examples of input to the AI include multiple response candidate texts, emotion labels, and emotion scores (e.g., [0.92, 0.85, 0.78]). The provision unit uses emotion estimation models (e.g., Transformer-based emotion classification model) and priority determination logic (e.g., priority mapping for each emotion label) to determine the output order of each voice content and generate order control signals. For example, for a “depression” label, encouraging words are output first; for an “excitement” label, words to calm down are output first; and for a “fatigue” label, words to help relax are output first. AI output includes a list of voice output order (e.g., first: “Please don't overdo it,” second: “Please make sure not to forget to take your medicine”), order label (e.g., “emotion priority”), and priority scores for each output. These outputs are passed to the speech synthesis unit and the voice is played in order. Unlike conventional order-non-considering voice output or manual order adjustment by humans, this non-conventional computer processing by AI—emotion estimation, priority determination, and order optimization—brings technical effects such as rapid and appropriate voice provision according to emotional state, improved user experience, and personalized dialogue control. Application fields include elderly monitoring robots, remote dialogue support, automatic multi-topic voice guidance in care settings, and support for people with disabilities.

The provision unit can adjust the volume and tone of the provided voice according to the environment of the elderly person. For example, when the surroundings of the elderly person are noisy, the provision unit can increase the volume of the voice. When the surroundings are quiet, the provision unit can provide the voice at an appropriate volume. Furthermore, the provision unit can adjust the tone of the voice according to the environment of the elderly person. This enables the provision of voice with a volume and tone appropriate to the environment. Specifically, the provision unit receives environmental sound level data (e.g., in dB SPL, 60-90 dB) obtained from environmental sound sensors (e.g., microphones, noise meters) and hearing profile (e.g., high-frequency attenuation, low-frequency sensitivity, hearing level in dB SPL) obtained from the user profile management unit as input. Examples of input to the AI include environmental sound level (e.g., 75 dB), voice waveform data (e.g., 16 kHz, PCM data), and hearing profile vector (e.g., [high-frequency −10 dB, mid-frequency −5 dB, low-frequency 0 dB]). The provision unit applies volume adjustment algorithms (e.g., automatic gain control, normalization), equalizer processing (e.g., high-frequency boost, low-frequency cut), and AI-based environment-adaptive voice optimization models (e.g., neural network-based volume and tone recommendation estimation), and automatically increases the volume (e.g., +10 dB correction) when the environment is noisy, or adjusts to an appropriate volume (e.g., 60 dB) when it is quiet. For tone, prosody control parameters (e.g., F0, speech rate, energy) are dynamically adjusted according to the spectral distribution of environmental sounds and the user's hearing characteristics. AI output includes adjusted voice waveform data (e.g., 16 kHz, PCM data, volume 80 dB SPL), recommended volume value (e.g., 80 dB SPL), and tone control parameters (e.g., F0=160 Hz, speech rate=0.8×). These outputs are provided to the elderly person via speakers or bone conduction devices. Unlike conventional fixed-volume and fixed-tone playback or manual adjustment by humans, this non-conventional computer processing by AI—environmental sound and hearing-adaptive voice optimization and real-time control—brings technical effects such as improved audibility, comfort, individual optimization, and enhanced accessibility for people with hearing loss. Application fields include elderly dialogue robots, remote voice guidance, automatic voice provision in care settings, support for people with disabilities, and hearing compensation-type automatic response systems.

The provision unit can adjust the level of detail of the provided voice according to the content spoken by the elderly person. For example, when the elderly person brings up an important topic, the provision unit can provide detailed information. When the elderly person brings up everyday topics, the provision unit can provide concise information. Furthermore, when the elderly person brings up an urgent topic, the provision unit can quickly provide detailed information. This enables the provision of voice with a level of detail appropriate to the content spoken. Specifically, the provision unit receives text data (e.g., “I want to go to the hospital,” “I forgot to take my medicine,” “The weather is nice today”), topic classification labels (e.g., “urgent,” “health,” “daily”), and topic importance scores (e.g., [0.98, 0.85, 0.60]) from the verbalization unit or response unit. Examples of input to the AI include text such as “I forgot to take my medicine,” topic classification label “health,” and importance score 0.85. The provision unit uses topic classification algorithms (e.g., Transformer-based topic classification model, rule-based classifier) and level-of-detail control logic (e.g., information amount adjustment according to importance score) to determine the level of detail of the voice content (e.g., detailed, concise, urgent detailed), and adds level-of-detail parameters to the content generation algorithm. For example, for urgent topics, detailed explanations and cautions (e.g., “When going to the hospital, be sure to bring your insurance card”) are included; for daily topics, concise responses (e.g., “It's sunny today”) are generated. AI output includes voice content text, level-of-detail label (e.g., “detailed,” “concise,” “urgent detailed”), and content priority score. These outputs are passed to the speech synthesis unit and used for voice output processing. Unlike conventional fixed-level-of-detail responses or manual adjustment by humans, this non-conventional computer processing by AI—topic classification, level-of-detail control, and real-time optimization—brings technical effects such as optimized information transmission, rapid response to urgency and importance, and improved user experience. Application fields include elderly monitoring robots, remote dialogue support, automatic guidance of important matters in care settings, and support for people with disabilities.

The monitoring unit can estimate the emotion of the elderly person and adjust the frequency of monitoring based on the estimated emotion. For example, when the elderly person is feeling down, the monitoring unit can increase the frequency of monitoring. When the elderly person is excited, the monitoring unit can decrease the frequency of monitoring. Furthermore, when the elderly person is tired, the monitoring unit can perform monitoring at a moderate frequency. This enables monitoring to be performed at a frequency appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the monitoring unit inputs the elderly person's utterance text received from the speech recognition unit or dialogue management unit (e.g., “I haven't been feeling well lately,” “I'm tired today”), voice feature quantities (e.g., F0, speech rate, energy), and past emotion estimation history vectors (e.g., the last five emotion labels and scores) as a multidimensional tensor into an emotion estimation model (e.g., Transformer-based emotion classification model, convolutional neural network, recurrent neural network). This emotion estimation model, trained by supervised learning, outputs emotion labels such as “sadness,” “excitement,” “fatigue,” “joy,” etc. Examples of input to the AI include text such as “I'm tired today,” voice feature vectors (e.g., F0=170 Hz, speech rate=0.7×, energy=low), and past emotion history (e.g., previous “fatigue”). AI output includes emotion labels (e.g., “sadness”), emotion scores (e.g., 0.91), and emotion distribution (e.g., sadness 0.91, joy 0.04, neutral 0.05). The monitoring unit uses monitoring frequency control algorithms (e.g., state transition tables, reinforcement learning-based frequency optimization models) to dynamically adjust the timing of vital measurements such as heart rate, body temperature, and activity level according to the estimated emotion label. For example, for a “sadness” label, the frequency is increased from once per hour to once every 15 minutes; for an “excitement” label, the frequency is decreased to once every two hours; and for a “fatigue” label, the normal frequency of once per hour is maintained. Examples of AI output include the next measurement timing (e.g., 15 minutes later), frequency control signal (e.g., high-frequency mode), and metadata with emotion labels. These outputs are passed to the vital sensor control unit and data recording unit, and the measurement frequency is changed in real time. Unlike conventional fixed-interval measurement or manual frequency adjustment by humans, this non-conventional computer processing by AI—emotion-adaptive frequency control and real-time emotion estimation—brings technical effects such as early detection of health risks during emotional fluctuations, optimization of measurement burden, and personalized health management for each user. Application fields include elderly monitoring robots, remote health management, emotion-adaptive vital monitoring in care settings, support for people with disabilities, and home medical support requiring mental care.

The monitoring unit can improve the accuracy of monitoring by referring to the past health data of the elderly person. For example, the monitoring unit can refer to past heart rate data of the elderly person to improve monitoring accuracy. The monitoring unit can also refer to past body temperature data to improve monitoring accuracy. Furthermore, the monitoring unit can refer to past health conditions to improve monitoring accuracy. This enables improved monitoring accuracy by referring to past health data. Specifically, the monitoring unit obtains time-series data such as heart rate vectors for the past week to month (e.g., [72, 74, 76, 80, 78, . . . ]), body temperature vectors (e.g., [36.5, 36.7, 36.8, 37.0, . . . ]), and abnormality judgment history (e.g., time and content of abnormal event occurrence) accumulated in local memory or cloud databases. The monitoring unit inputs these history data into AI-based anomaly detection models (e.g., LSTM-based time-series anomaly detection model, autoregressive neural network, Bayesian estimation model) to individually optimize thresholds and trends for normal and abnormal patterns. Examples of input to the AI include heart rate vectors for the past 30 days, body temperature vectors, and abnormal event flags (e.g., 1 or 0). AI output includes individually optimized anomaly detection thresholds (e.g., heart rate upper limit 115 bpm, body temperature upper limit 37.8° C.), anomaly scores (e.g., 0.97), and predicted abnormal occurrence probability (e.g., 0.12). The monitoring unit uses these output values to dynamically correct the judgment logic for real-time measurement values and realize personalized monitoring based on past health conditions. For example, for elderly persons with a tendency toward higher body temperature than normal, the body temperature abnormality judgment threshold is individually adjusted. Unlike conventional uniform threshold judgment or manual history reference by humans, this non-conventional computer processing by AI—history reference, individual optimization, and dynamic threshold correction—brings technical effects such as improved monitoring accuracy, reduced false detections and missed detections, and rapid response to changes in health condition for each user. Application fields include elderly monitoring robots, remote health management, individually optimized vital monitoring in care settings, support for people with disabilities, and chronic disease management.

The monitoring unit can optimize the timing of monitoring according to the lifestyle rhythm of the elderly person. For example, when the elderly person is a morning type, the monitoring unit can perform monitoring in the morning hours. When the elderly person is a night type, the monitoring unit can perform monitoring in the evening hours. Furthermore, the monitoring unit can perform monitoring at the optimal timing according to the lifestyle rhythm of the elderly person. This enables monitoring to be performed at timing appropriate to the lifestyle rhythm. Specifically, the monitoring unit extracts the daily activity pattern of the elderly person from the user profile management unit and lifestyle rhythm estimation algorithms (e.g., time-stamped activity data, sleep/wake time records, meal/medication time logs). Examples of input to the AI include wake-up and bedtime vectors for the past week (e.g., [6:30, 22:00]), time-series activity data (e.g., hourly step count and exercise intensity), and lifestyle event labels (e.g., “breakfast,” “bath”). The monitoring unit inputs these data into time-series clustering models or LSTM-based lifestyle rhythm estimation models and outputs lifestyle rhythm labels (e.g., “morning type”), optimal monitoring timing lists (e.g., 7:00, 12:00, 19:00), and recommended measurement intervals (e.g., every 3 hours). The monitoring unit automatically adjusts the scheduling of vital measurements and health checks based on the output timing information. For example, for a “morning type” label, measurements are focused at 7 a.m., 12 p.m., and 7 p.m.; for a “night type” label, measurements are performed at 12 p.m., 6 p.m., and 10 p.m. Unlike conventional fixed-time measurement or manual scheduling by humans, this non-conventional computer processing by AI—lifestyle rhythm-adaptive timing optimization and real-time scheduling—brings technical effects such as optimized measurement timing, flexible response to lifestyle patterns, and reduced user burden. Application fields include elderly monitoring robots, remote health management, lifestyle rhythm-adaptive vital monitoring in care settings, support for people with disabilities, and sleep disorder management.

The monitoring unit can estimate the emotion of the elderly person and determine the priority of monitoring based on the estimated emotion. For example, when the elderly person is feeling down, the monitoring unit can increase the priority of monitoring. When the elderly person is excited, the monitoring unit can decrease the priority of monitoring. Furthermore, when the elderly person is tired, the monitoring unit can perform monitoring with moderate priority. This enables monitoring to be performed with a priority appropriate to the emotion of the elderly person. Emotion estimation is realized using an emotion estimation function, for example, by employing an emotion engine or generative AI. Generative AI may include text generation AI (such as LLMs) or multimodal generative AI, but is not limited to these examples. Specifically, the monitoring unit inputs utterance text received from the speech recognition unit or dialogue management unit, voice feature quantities, and past emotion estimation history vectors into an emotion estimation model (e.g., Transformer-based emotion classification model), and outputs emotion labels (e.g., “depression,” “excitement,” “fatigue”) and emotion scores (e.g., 0.92). Examples of input to the AI include text such as “I'm tired today,” voice feature vectors, and past emotion history (e.g., results of the last five emotion estimations). The monitoring unit calculates priority scores for each monitoring item (e.g., heart rate: 0.95, body temperature: 0.90, activity level: 0.80) according to the estimated emotion label, and uses priority determination logic (e.g., weighted scoring, priority ranking) to determine the measurement order and key items. For example, for a “depression” label, the priority of heart rate and body temperature measurement is increased and the priority of activity level is decreased; for an “excitement” label, the priority of activity level is increased. AI output includes a list of monitoring items with priority (e.g., 1st: heart rate, 2nd: body temperature, 3rd: activity level), priority scores, and emotion labels. These outputs are passed to the measurement scheduler and data recording unit, and the priority is reflected in real time. Unlike conventional fixed-priority measurement or manual priority adjustment by humans, this non-conventional computer processing by AI—emotion-adaptive priority control and real-time emotion estimation—brings technical effects such as prevention of missing important items, optimization of measurement efficiency, and personalized health management for each user. Application fields include elderly monitoring robots, remote health management, emotion-adaptive vital monitoring in care settings, support for people with disabilities, and home medical support requiring mental care.

The monitoring unit can customize the content of monitoring by referring to the environmental data of the elderly person. For example, when the living environment of the elderly person is a cold region, the monitoring unit can strengthen the monitoring of room temperature. When the living environment is hot and humid, the monitoring unit can strengthen the monitoring of humidity. Furthermore, the monitoring unit can customize the monitoring items according to the living environment of the elderly person. This enables monitoring to be performed with content appropriate to the environmental data. Specifically, the monitoring unit receives living environment data (e.g., room temperature 5-35° C., humidity 20-90%, CO2 concentration, illumination level) obtained from environmental sensors (e.g., temperature sensor, humidity sensor, CO2 sensor, illumination sensor) and residential area information (e.g., cold region, hot and humid region, urban area, mountainous area) obtained from the user profile management unit as input. Examples of input to the AI include room temperature data (e.g., 10° C.), humidity data (e.g., 85%), area label (e.g., “cold region”), and past environmental abnormality history. The monitoring unit uses environment-adaptive monitoring item selection algorithms (e.g., rule-based dictionary, decision tree, neural network with attention mechanism) to automatically determine the measurement item list (e.g., room temperature, humidity, CO2, illumination) and the measurement frequency and threshold for each item. For example, for a cold region label, the frequency of room temperature measurement is increased to every 15 minutes and the low temperature warning threshold is set to 18° C.; for a hot and humid region, the frequency of humidity measurement is strengthened and heatstroke risk judgment is added. AI output includes a customized monitoring item list, measurement frequency and threshold for each item, and environmental abnormality warning flags. These outputs are passed to the sensor control unit and data recording unit, and the monitoring content is changed in real time. Unlike conventional uniform item measurement or manual environment setting by humans, this non-conventional computer processing by AI—environment-adaptive item selection and real-time optimization—brings technical effects such as rapid response to environmental risks, optimization of measurement efficiency, and personalized health management for each user. Application fields include elderly monitoring robots, remote health management, environment-adaptive vital monitoring in care settings, support for people with disabilities, and support for prevention of heatstroke and hypothermia.

The monitoring unit can adjust the monitoring items according to the spoken content of the elderly person. For example, if the elderly person complains of feeling unwell, the monitoring unit can enhance the monitoring of health conditions. If the elderly person complains of lack of exercise, the monitoring unit can enhance the monitoring of activity levels. Furthermore, if the elderly person complains of sleep deprivation, the monitoring unit can enhance the monitoring of sleep conditions. In this way, monitoring can be performed for items corresponding to the spoken content. Specifically, the monitoring unit passes the utterance text (e.g., “I have not been feeling well lately,” “I haven't been able to exercise much,” “I can't sleep well”) received from the verbalization unit or dialogue management unit to the natural language processing unit, and uses topic classification algorithms (e.g., Transformer-based topic classification models, rule-based classifiers) and keyword extraction algorithms (e.g., TF-IDF, neural networks with attention mechanisms) to automatically extract monitoring enhancement items (e.g., health condition, activity level, sleep condition). Examples of AI input include the text “I have not been feeling well lately,” past utterance history vectors, and topic classification labels (e.g., “health”). The monitoring unit dynamically adjusts the measurement frequency and recording items of vital sensors (e.g., heart rate, body temperature), activity sensors (e.g., accelerometers, pedometers), and sleep sensors (e.g., bed sensors, sleep apps) according to the extracted items. For example, in the case of a “feeling unwell” label, the measurement frequency of heart rate and body temperature is increased; in the case of a “lack of exercise” label, the recording of activity levels is enhanced; and in the case of a “sleep deprivation” label, detailed recording of sleep conditions is added. AI output includes a list of enhancement items, measurement frequency and detail level for each item, and topic labels. These outputs are passed to the sensor control unit and data recording unit, and the monitoring content is changed in real time. Unlike conventional fixed item measurement or manual item adjustment by humans, this unconventional computer processing by AI—topic-adaptive item selection and real-time optimization—provides technical effects such as rapid response to user complaints, optimization of measurement efficiency, and personalized health management. Application fields include elderly monitoring robots, remote health management, complaint-adaptive vital monitoring in care settings, support for persons with disabilities, and support for sleep disorder and lack of exercise.

The notification unit can estimate the emotion of the elderly person and adjust the content of the notification based on the estimated emotion. For example, if the elderly person is feeling down, the notification unit can send a notification to the family including an encouraging message. If the elderly person is excited, the notification unit can send a notification including a calming message. Furthermore, if the elderly person is tired, the notification unit can send a notification including a message encouraging rest. In this way, notifications can be made with content corresponding to the emotion. Emotion estimation is realized using, for example, an emotion engine or generative AI with emotion estimation functions. Generative AI may include text generation AI (e.g., LLM) or multimodal generative AI, but is not limited to these examples. Specifically, the notification unit inputs the elderly person's utterance text (e.g., “I haven't been feeling well lately,” “I'm tired today”), voice features (e.g., F0, speech rate, energy), and past emotion estimation history vectors (e.g., emotion labels and scores for the last five times) received from the speech recognition unit or dialogue management unit as multidimensional tensors into an emotion estimation model (e.g., Transformer-based emotion classification model, convolutional neural network, recurrent neural network). The emotion estimation model, trained by supervised learning, outputs emotion labels such as “sadness,” “excitement,” “fatigue,” “joy,” etc. Examples of AI input include the text “I'm tired today,” voice feature vectors (e.g., F0=170 Hz, speech rate=0.7×, energy=low), and past emotion history (e.g., previous “fatigue”). AI output includes emotion labels (e.g., “sadness”), emotion scores (e.g., 0.91), and emotion distributions (e.g., sadness 0.91, joy 0.04, neutral 0.05). The notification unit dynamically switches vocabulary selection dictionaries, expression templates, and style transformation rules for the notification content generation algorithm (e.g., large language models, template-based generation, rule-based expression optimization) according to the estimated emotion label. For example, in the case of a “sadness” label, an empathetic message such as “The user seems to be feeling down lately. Please contact them with encouragement” is included for the family; in the case of an “excitement” label, a calm expression such as “The user seems a bit excited. Please respond to help them calm down” is selected; and in the case of a “fatigue” label, a message such as “The user seems tired, so please encourage them to rest” is generated. Examples of AI input include the text “I'm tired today,” emotion label “fatigue,” and past conversation history vectors, with output such as “The user seems tired. Family members are encouraged to help them rest.” The generated notification content is passed to the notification system for the family or medical institution and sent via email, SMS, app notification, etc. Unlike conventional fixed-message notifications or manual content adjustment by humans, this unconventional computer processing by AI—multidimensional feature analysis, emotion estimation, and expression optimization—provides technical effects such as high-precision emotion-adaptive notification content generation, promotion of rapid and appropriate responses by family and caregivers, and personalized notifications for each user. Application fields include elderly monitoring robots, remote monitoring systems, emotion-adaptive automatic notification in care settings, support for persons with disabilities, and emotion-sensitive emergency notification systems.

The notification unit can refer to the past health data of the elderly person to improve the accuracy of notifications. For example, the notification unit can refer to the elderly person's past heart rate data to improve notification accuracy. The notification unit can also refer to past body temperature data to improve notification accuracy. Furthermore, the notification unit can refer to the elderly person's past health condition to improve notification accuracy. In this way, referring to past health data improves the accuracy of notifications. Specifically, the notification unit acquires time-series data such as heart rate vectors for the past week to month (e.g., [72, 74, 76, 80, 78, . . . ]), body temperature vectors (e.g., [36.5, 36.7, 36.8, 37.0, . . . ]), and abnormality judgment history (e.g., timestamps and details of abnormal events) stored in local memory or a cloud database. The notification unit inputs these historical data into an AI-based anomaly detection model (e.g., LSTM-based time-series anomaly detection model, autoregressive neural network, Bayesian estimation model) to individually optimize thresholds and trends for normal and abnormal patterns. Examples of AI input include heart rate vectors for the past 30 days, body temperature vectors, and abnormal event flags (e.g., 1 or 0). AI output includes individually optimized anomaly detection thresholds (e.g., heart rate upper limit 115 bpm, body temperature upper limit 37.8° C.), anomaly scores (e.g., 0.97), and predicted anomaly occurrence probabilities (e.g., 0.12). The notification unit uses these output values to dynamically correct the judgment logic for real-time measurements, enabling personalized notification judgments based on past health conditions. For example, for an elderly person with a tendency toward higher body temperature, the body temperature anomaly judgment threshold is individually adjusted to reduce false alarms and missed detections. Examples of AI output include notification content such as “Heart rate has deviated significantly from past trends. Please check immediately,” and notification priority labels with anomaly scores (e.g., high priority). These outputs are passed to the notification system for the family or medical institution, and notifications are automatically sent according to the urgency. Unlike conventional uniform threshold judgments or manual history reference by humans, this unconventional computer processing by AI—history reference, individual optimization, and dynamic threshold correction—provides technical effects such as improved notification accuracy, reduced false detections and missed detections, and rapid response to changes in each user's health condition. Application fields include elderly monitoring robots, remote health management, individually optimized automatic notification in care settings, support for persons with disabilities, and chronic disease management.

The notification unit can optimize the timing of notifications according to the lifestyle rhythm of the elderly person. For example, if the elderly person is a morning type, the notification unit can send notifications in the morning hours. If the elderly person is a night type, the notification unit can send notifications in the evening hours. Furthermore, the notification unit can send notifications at optimal times according to the lifestyle rhythm of the elderly person. In this way, notifications can be sent at timings corresponding to the lifestyle rhythm. Specifically, the notification unit extracts the daily activity patterns of the elderly person from the user profile management unit and lifestyle rhythm estimation algorithms (e.g., time-stamped activity data, sleep/wake time records, meal/medication time logs). Examples of AI input include vectors of wake-up and bedtimes for the past week (e.g., [6:30, 22:00]), time-series activity data (e.g., hourly steps or exercise intensity), and lifestyle event labels (e.g., “breakfast,” “bath”). The notification unit inputs these data into time-series clustering models or LSTM-based lifestyle rhythm estimation models to output lifestyle rhythm labels such as “morning type,” “night type,” or “irregular type.” AI output includes lifestyle rhythm labels (e.g., “morning type”), optimal notification timing lists (e.g., 7:00, 12:00, 19:00), and recommended notification intervals (e.g., every 3 hours). The notification unit automatically adjusts notification scheduling based on the output timing information. For example, for a “morning type” label, notifications are focused at 7:00 a.m., 12:00 p.m., and 7:00 p.m.; for a “night type” label, notifications are sent at 12:00 p.m., 6:00 p.m., and 10:00 p.m. Examples of AI output include content such as “Health status notifications will be sent in the morning hours,” and notification timing control signals (e.g., notification at 7:00). Unlike conventional fixed-time notifications or manual scheduling by humans, this unconventional computer processing by AI—lifestyle rhythm-adaptive timing optimization and real-time scheduling—provides technical effects such as optimization of notification timing, flexible adaptation to lifestyle patterns, and reduction of user burden. Application fields include elderly monitoring robots, remote health management, lifestyle rhythm-adaptive automatic notification in care settings, support for persons with disabilities, and sleep disorder management.

The notification unit can estimate the emotion of the elderly person and determine the priority of notifications based on the estimated emotion. For example, if the elderly person is feeling down, the notification unit can increase the priority of the notification. If the elderly person is excited, the notification unit can lower the priority of the notification. Furthermore, if the elderly person is tired, the notification unit can send notifications with moderate priority. In this way, notifications can be sent with priorities corresponding to the emotion. Emotion estimation is realized using, for example, an emotion engine or generative AI with emotion estimation functions. Generative AI may include text generation AI (e.g., LLM) or multimodal generative AI, but is not limited to these examples. Specifically, the notification unit inputs utterance text, voice features, and past emotion estimation history vectors received from the speech recognition unit or dialogue management unit into an emotion estimation model (e.g., Transformer-based emotion classification model) to output emotion labels (e.g., “depression,” “excitement,” “fatigue”) and emotion scores (e.g., 0.92). Examples of AI input include the text “I'm tired today,” voice feature vectors, and past emotion history (e.g., the last five emotion estimation results). The notification unit calculates notification priority scores (e.g., high: 0.95, medium: 0.80, low: 0.60) according to the estimated emotion label and determines the notification order and urgency using priority judgment logic (e.g., weighted scoring, priority ranking). For example, in the case of a “depression” label, the notification priority is increased and immediate notification is performed; in the case of an “excitement” label, the priority is lowered and situation monitoring is prioritized; and in the case of a “fatigue” label, normal notification is performed. AI output includes notification content with priority (e.g., “Please check immediately”), priority scores, and emotion labels. These outputs are passed to the notification scheduler and notification system, and priorities are reflected in real time. Unlike conventional fixed-priority notifications or manual priority adjustment by humans, this unconventional computer processing by AI—emotion-adaptive priority control and real-time emotion estimation—provides technical effects such as prevention of missing important items, optimization of notification efficiency, and personalized monitoring for each user. Application fields include elderly monitoring robots, remote health management, emotion-adaptive automatic notification in care settings, support for persons with disabilities, and home medical support requiring mental care.

The notification unit can refer to the environmental data of the elderly person to customize the content of notifications. For example, if the elderly person's living environment is in a cold region, the notification unit can send notifications including information about room temperature. If the living environment is hot and humid, the notification unit can send notifications including information about humidity. Furthermore, the notification unit can customize the notification content according to the living environment of the elderly person. In this way, notifications can be sent with content corresponding to environmental data. Specifically, the notification unit receives living environment data (e.g., room temperature 5-35° C., humidity 20-90%, CO2 concentration, illuminance level) obtained from environmental sensors (e.g., temperature sensor, humidity sensor, CO2 sensor, illuminance sensor) and residential area information (e.g., cold region, hot and humid region, urban area, mountainous area) obtained from the user profile management unit as input. Examples of AI input include room temperature data (e.g., 10° C.), humidity data (e.g., 85%), region label (e.g., “cold region”), and past environmental abnormality history. The notification unit uses an environment-adaptive notification content generation algorithm (e.g., rule-based dictionary, decision tree, neural network with attention mechanism) to automatically determine the notification content list (e.g., room temperature information, humidity information, CO2 information, illuminance information) and the notification frequency and thresholds for each item. For example, in the case of a cold region label, the notification content is “Room temperature has dropped to 10° C. Please check the heating”; in the case of a hot and humid region, the content is “Humidity has risen to 85%. Please be careful of heatstroke.” AI output includes customized notification content text, notification frequency and thresholds for each item, and environmental abnormality warning flags. These outputs are passed to the notification system for family and caregivers, and notifications are sent in real time according to environmental risks. Unlike conventional uniform item notifications or manual environmental settings by humans, this unconventional computer processing by AI—environment-adaptive content selection and real-time optimization—provides technical effects such as rapid response to environmental risks, optimization of notification efficiency, and personalized monitoring for each user. Application fields include elderly monitoring robots, remote health management, environment-adaptive automatic notification in care settings, support for persons with disabilities, and support for prevention of heatstroke and hypothermia.

The notification unit can adjust the notification items according to the spoken content of the elderly person. For example, if the elderly person complains of feeling unwell, the notification unit can send notifications regarding health condition. If the elderly person complains of lack of exercise, the notification unit can send notifications regarding activity level. Furthermore, if the elderly person complains of sleep deprivation, the notification unit can send notifications regarding sleep condition. In this way, notifications can be sent for items corresponding to the spoken content. Specifically, the notification unit passes the utterance text (e.g., “I have not been feeling well lately,” “I haven't been able to exercise much,” “I can't sleep well”) received from the verbalization unit or dialogue management unit to the natural language processing unit, and uses topic classification algorithms (e.g., Transformer-based topic classification models, rule-based classifiers) and keyword extraction algorithms (e.g., TF-IDF, neural networks with attention mechanisms) to automatically extract notification enhancement items (e.g., health condition, activity level, sleep condition). Examples of AI input include the text “I have not been feeling well lately,” past utterance history vectors, and topic classification labels (e.g., “health”). The notification unit reflects the measurement values from vital sensors (e.g., heart rate, body temperature), activity sensors (e.g., accelerometers, pedometers), and sleep sensors (e.g., bed sensors, sleep apps) in the notification content and dynamically switches notification templates according to the extracted items. For example, in the case of a “feeling unwell” label, the content generated is “There has been a complaint of feeling unwell recently. The latest heart rate and body temperature values are as follows”; in the case of a “lack of exercise” label, the content generated is “Activity level has decreased. Please support exercise”; and in the case of a “sleep deprivation” label, the content generated is “Problems have been observed in sleep condition. Please check.” AI output includes a list of enhancement items, notification content text for each item, and topic labels. These outputs are passed to the notification system for family and caregivers, and the notification content is changed in real time. Unlike conventional fixed item notifications or manual item adjustment by humans, this unconventional computer processing by AI—topic-adaptive item selection and real-time optimization—provides technical effects such as rapid response to user complaints, optimization of notification efficiency, and personalized monitoring. Application fields include elderly monitoring robots, remote health management, complaint-adaptive automatic notification in care settings, support for persons with disabilities, and support for sleep disorder and lack of exercise.

The system according to the embodiment is not limited to the examples described above, and various modifications are possible, for example, as follows.

The acquisition unit can measure the activity level of the elderly person and record daily exercise amounts. For example, the acquisition unit can measure the distance and time of walks taken by the elderly person and record daily exercise amounts. The acquisition unit can also record the content and time of household chores performed by the elderly person. Furthermore, the acquisition unit can record the content and progress of rehabilitation performed by the elderly person. In this way, the daily activity level of the elderly person can be understood and utilized for health management.

The determination unit can record the meal content of the elderly person and evaluate nutritional balance. For example, the determination unit can record the content of meals consumed by the elderly person and evaluate the balance of calories and nutrients. The determination unit can also record the amount of water consumed by the elderly person and evaluate whether appropriate hydration is being provided. Furthermore, the determination unit can record the types and amounts of medication taken by the elderly person and support medication management. In this way, meals, hydration, and medication management for the elderly person can be centrally managed.

The provision unit can propose daily activities based on the hobbies and interests of the elderly person. For example, if the elderly person is interested in gardening, the provision unit can propose plant care methods according to the season. If the elderly person is interested in cooking, the provision unit can also propose simple recipes. Furthermore, if the elderly person is interested in handicrafts, the provision unit can propose new handicraft projects. In this way, activities corresponding to the hobbies and interests of the elderly person can be proposed, enriching daily life.

The acquisition unit can monitor the sleep patterns of the elderly person and evaluate sleep quality. For example, the acquisition unit can record the bedtime and wake-up time of the elderly person and understand the rhythm of sleep. The acquisition unit can also record movements during sleep and evaluate the depth and quality of sleep. Furthermore, the acquisition unit can record snoring and breathing conditions and evaluate the risk of sleep apnea syndrome. In this way, information for improving the sleep quality of the elderly person can be provided.

The provision unit can estimate the emotion of the elderly person and provide appropriate music based on the estimated emotion. For example, if the elderly person is feeling down, the provision unit can provide relaxing music. If the elderly person is excited, the provision unit can also provide calming music. Furthermore, if the elderly person is tired, the provision unit can provide refreshing music. In this way, music corresponding to the emotion of the elderly person can be provided to improve mood.

The determination unit can estimate the emotion of the elderly person and propose appropriate activities based on the estimated emotion. For example, if the elderly person is feeling down, the determination unit can propose a walk or light exercise. If the elderly person is excited, the determination unit can also propose relaxing yoga or meditation. Furthermore, if the elderly person is tired, the determination unit can propose rest or relaxing reading. In this way, activities corresponding to the emotion of the elderly person can be proposed to improve quality of life.

The notification unit can estimate the emotion of the elderly person and adjust the content of the notification based on the estimated emotion. For example, if the elderly person is feeling down, the notification unit can send a notification to the family including an encouraging message. If the elderly person is excited, the notification unit can also send a notification including a calming message. Furthermore, if the elderly person is tired, the notification unit can send a notification including a message encouraging rest. In this way, notifications can be made with content corresponding to the emotion.

The provision unit can estimate the emotion of the elderly person and adjust the content of the provided voice based on the estimated emotion. For example, if the elderly person is sad, the provision unit can provide encouraging words. If the elderly person is excited, the provision unit can also provide calming words. Furthermore, if the elderly person is tired, the provision unit can provide relaxing words. In this way, voice can be provided with content corresponding to the emotion of the elderly person.

The monitoring unit can estimate the emotion of the elderly person and adjust the frequency of monitoring based on the estimated emotion. For example, if the elderly person is feeling down, the monitoring unit can increase the frequency of monitoring. If the elderly person is excited, the monitoring unit can also decrease the frequency of monitoring. Furthermore, if the elderly person is tired, the monitoring unit can perform monitoring at a moderate frequency. In this way, monitoring can be performed at a frequency corresponding to the emotion of the elderly person.

The provision unit can estimate the emotion of the elderly person and adjust the order of the provided voice based on the estimated emotion. For example, if the elderly person is feeling down, the provision unit can provide encouraging words first. If the elderly person is excited, the provision unit can also provide calming words first. Furthermore, if the elderly person is tired, the provision unit can provide relaxing words first. In this way, voice can be provided in an order corresponding to the emotion of the elderly person.

The following is a brief explanation of the processing flow of Example of the Embodiment.
Step 1: The verbalization unit verbalizes the spoken content using speech recognition technology. For example, deep learning-based speech recognition technology or HMM (Hidden Markov Model)-based speech recognition technology can be used to accurately verbalize the spoken content of the elderly person.
Step 2: The response unit generates an appropriate response based on the content verbalized by the verbalization unit. For example, natural language generation technology or template-based response generation technology can be used to generate an appropriate response for the elderly person.
Step 3: The output unit outputs the response generated by the response unit by voice. For example, speech synthesis technology can be used to output the generated response as high-quality voice.
Step 4: The provision unit provides the voice output by the output unit to the elderly person. For example, the output voice can be provided at a volume that is easy for the elderly person to hear.
Step 5: The monitoring unit monitors the health condition of the elderly person based on the voice provided by the provision unit. For example, the heart rate and body temperature of the elderly person can be periodically measured to monitor health condition.
Step 6: The notification unit notifies a medical institution or family in case of emergency based on the health condition monitored by the monitoring unit. For example, if an abnormality is detected, notification can be automatically sent to a medical institution or family.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned verbalization unit, response unit, output unit, provision unit, monitoring unit, and notification unit is implemented by at least one of, for example, a smart device 14 and a data processing apparatus 12. For example, the verbalization unit is implemented by a processor 46 of the smart device 14 and verbalizes the spoken content of the elderly person using speech recognition technology. The response unit is implemented, for example, by a specific processing unit 290 of the data processing apparatus 12 and generates an appropriate response based on the verbalized content. The output unit is implemented, for example, by the processor 46 of the smart device 14 and outputs the generated response by voice. The provision unit is implemented, for example, by an output device 40 of the smart device 14 and provides the output voice to the elderly person. The monitoring unit monitors the health condition of the elderly person using, for example, a camera 42 or sensor of the smart device 14. The notification unit is implemented, for example, by the specific processing unit 290 of the data processing apparatus 12 and notifies a medical institution or family in case of emergency. The correspondence between each unit and the device or control unit is not limited to the examples described above and various modifications are possible.

### Second Embodiment

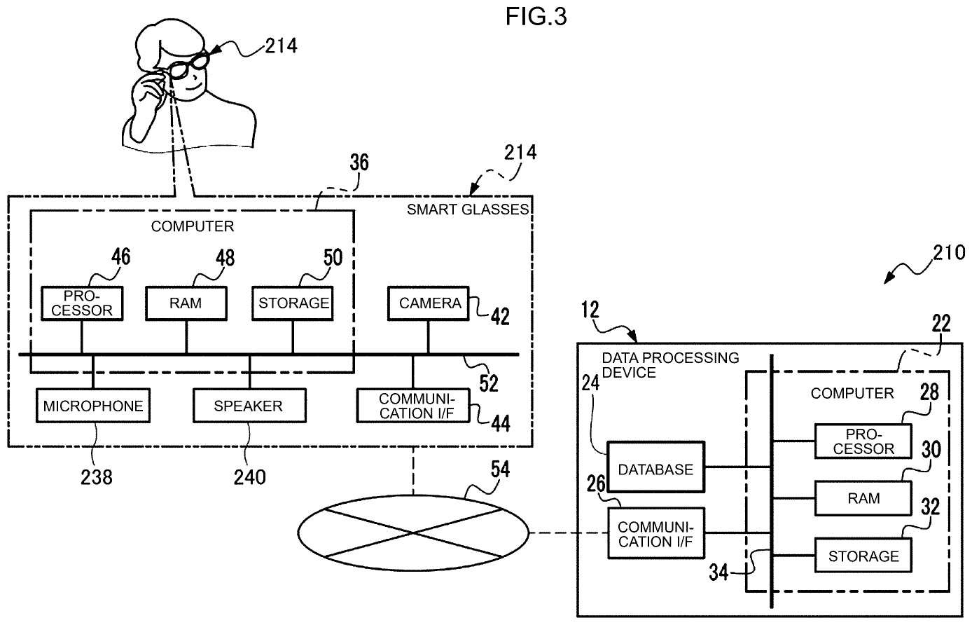

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

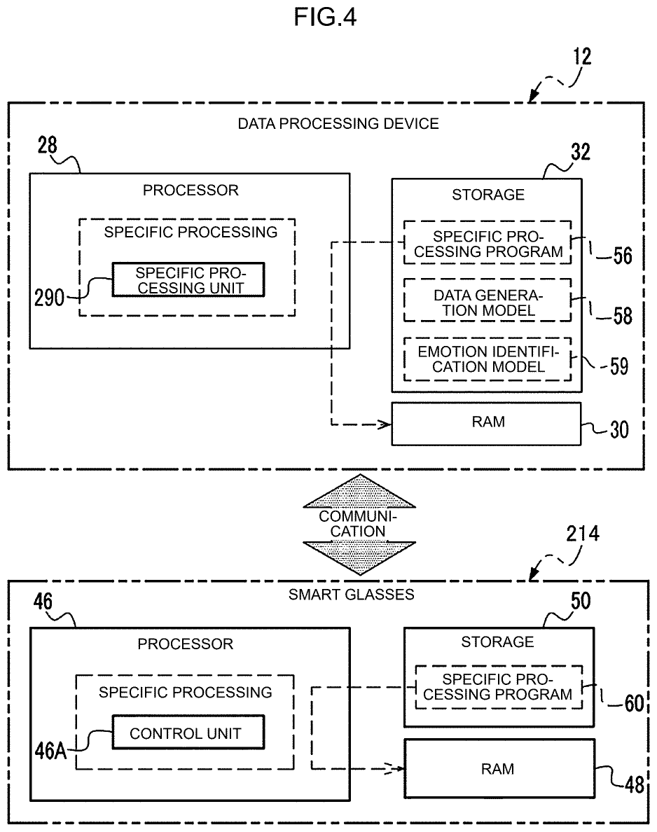

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned verbalization unit, response unit, output unit, provision unit, monitoring unit, and notification unit is implemented by at least one of, for example, smart glasses 214 and a data processing apparatus 12. For example, the verbalization unit is implemented by a processor 46 of the smart glasses 214 and verbalizes the spoken content of the elderly person using speech recognition technology. The response unit is implemented, for example, by a specific processing unit 290 of the data processing apparatus 12 and generates an appropriate response based on the verbalized content. The output unit is implemented, for example, by the processor 46 of the smart glasses 214 and outputs the generated response by voice. The provision unit is implemented, for example, by a speaker 240 of the smart glasses 214 and provides the output voice to the elderly person. The monitoring unit monitors the health condition of the elderly person using, for example, a camera 42 or sensor of the smart glasses 214. The notification unit is implemented, for example, by the specific processing unit 290 of the data processing apparatus 12 and notifies a medical institution or family in case of emergency. The correspondence between each unit and the device or control unit is not limited to the examples described above and various modifications are possible.

### Third Embodiment

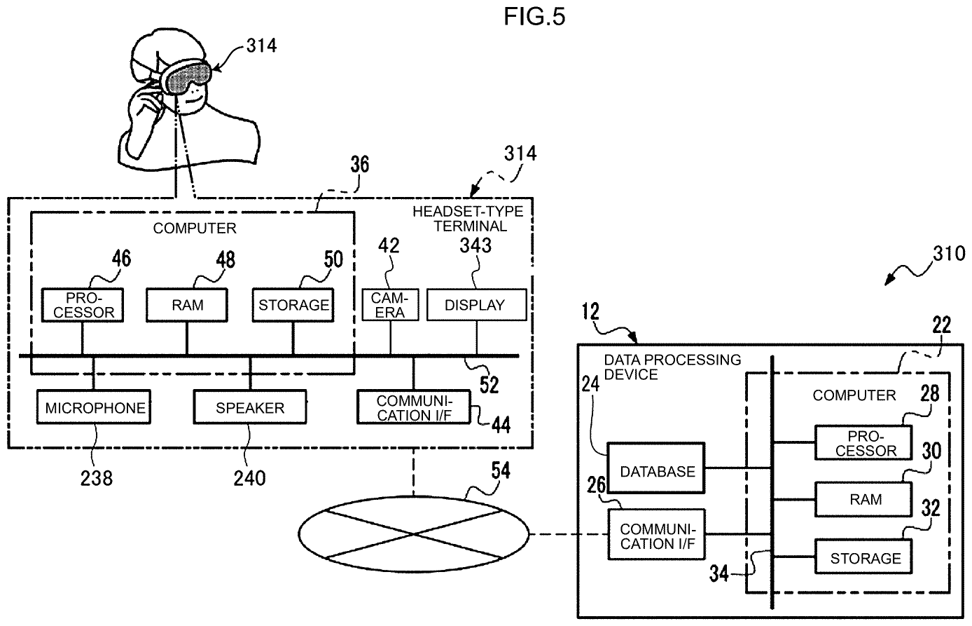

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

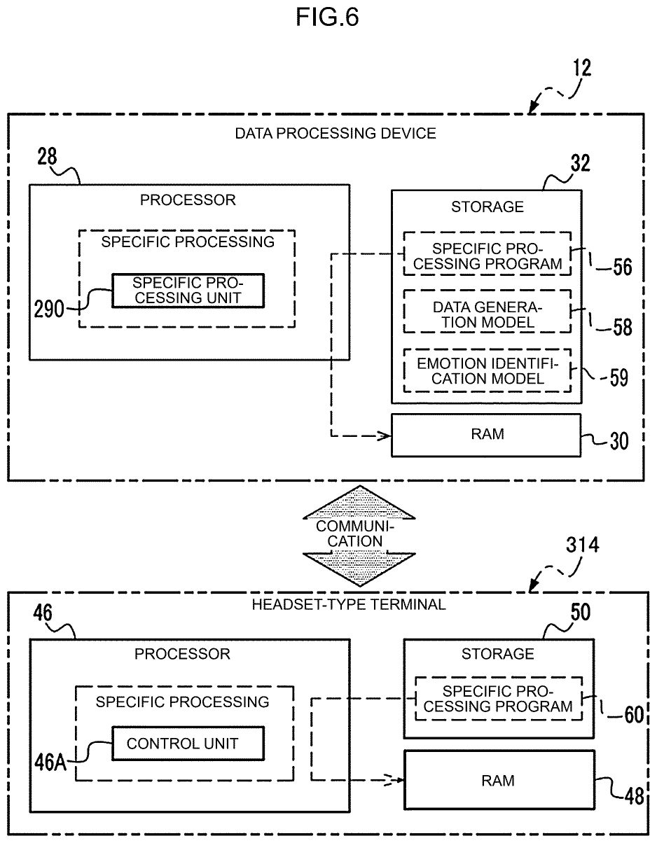

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned verbalization unit, response unit, output unit, provision unit, monitoring unit, and notification unit is implemented by at least one of, for example, a headset-type terminal 314 and a data processing apparatus 12. For example, the verbalization unit is implemented by a processor 46 of the headset-type terminal 314 and verbalizes the spoken content of the elderly person using speech recognition technology. The response unit is implemented, for example, by a specific processing unit 290 of the data processing apparatus 12 and generates an appropriate response based on the verbalized content. The output unit is implemented, for example, by the processor 46 of the headset-type terminal 314 and outputs the generated response by voice. The provision unit is implemented, for example, by a speaker 240 of the headset-type terminal 314 and provides the output voice to the elderly person. The monitoring unit monitors the health condition of the elderly person using, for example, a camera 42 or sensor of the headset-type terminal 314. The notification unit is implemented, for example, by the specific processing unit 290 of the data processing apparatus 12 and notifies a medical institution or family in case of emergency. The correspondence between each unit and the device or control unit is not limited to the examples described above and various modifications are possible.

### Fourth Embodiment

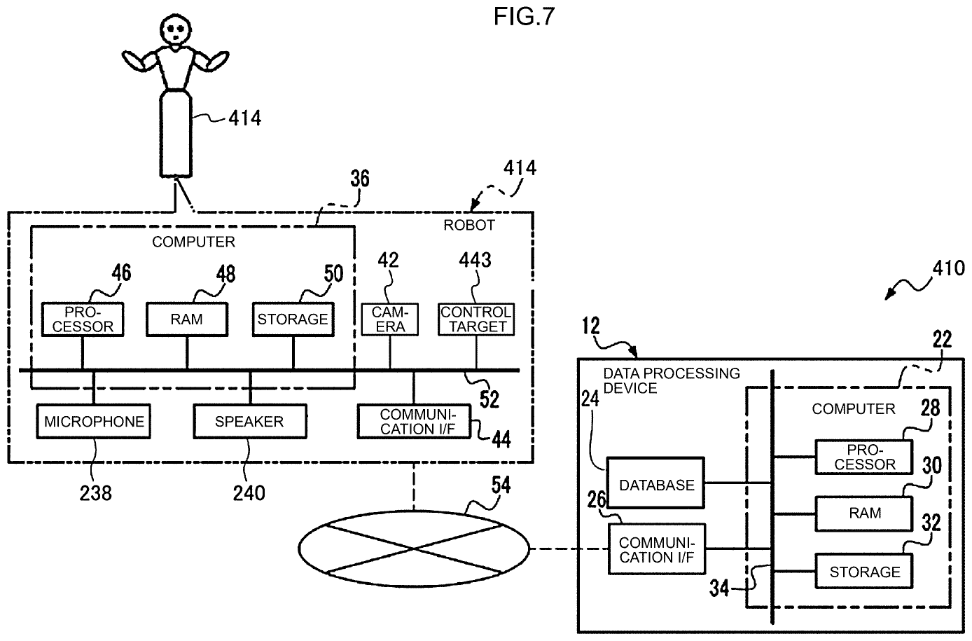

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

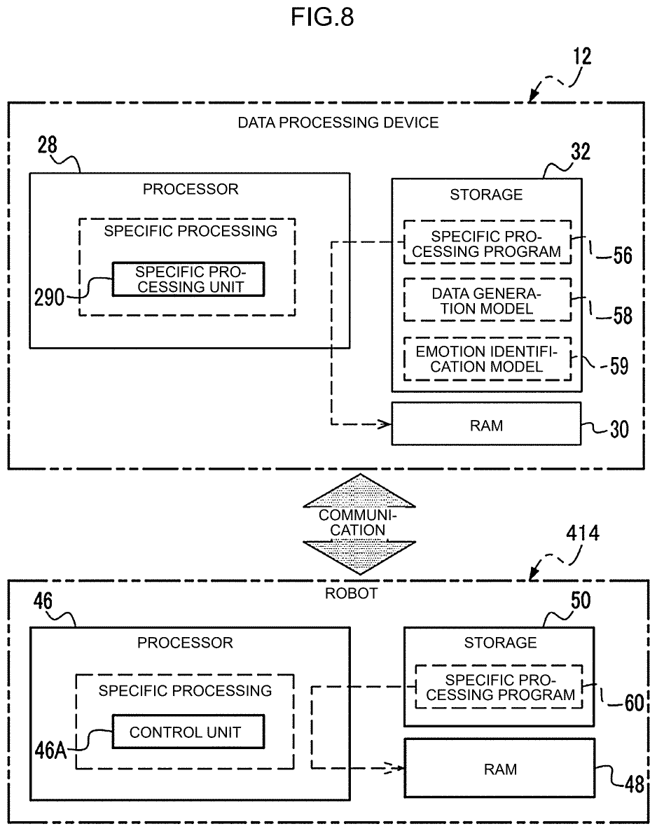

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned verbalization unit, response unit, output unit, provision unit, monitoring unit, and notification unit is implemented by at least one of, for example, a robot 414 and a data processing apparatus 12. For example, the verbalization unit is implemented by a processor 46 of the robot 414 and verbalizes the spoken content of the elderly person using speech recognition technology. The response unit is implemented, for example, by a specific processing unit 290 of the data processing apparatus 12 and generates an appropriate response based on the verbalized content. The output unit is implemented, for example, by the processor 46 of the robot 414 and outputs the generated response by voice. The provision unit is implemented, for example, by a speaker 240 of the robot 414 and provides the output voice to the elderly person. The monitoring unit monitors the health condition of the elderly person using, for example, a camera 42 or sensor of the robot 414. The notification unit is implemented, for example, by the specific processing unit 290 of the data processing apparatus 12 and notifies a medical institution or family in case of emergency. The correspondence between each unit and the device or control unit is not limited to the examples described above and various modifications are possible.

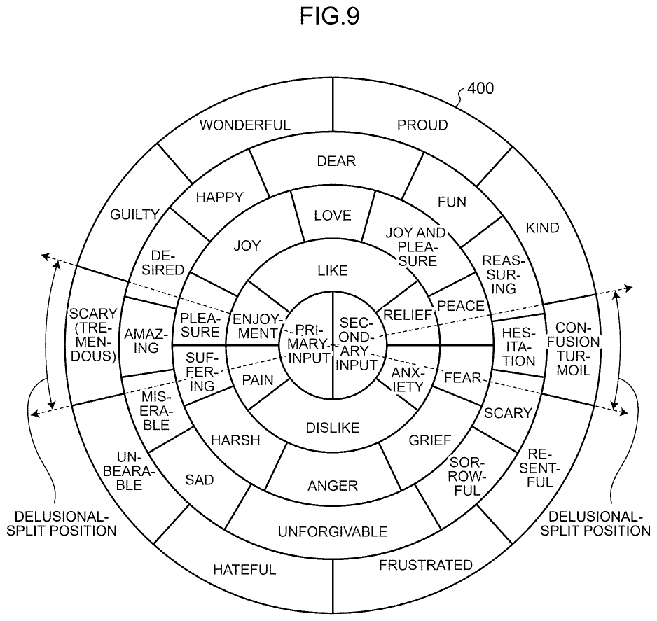

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

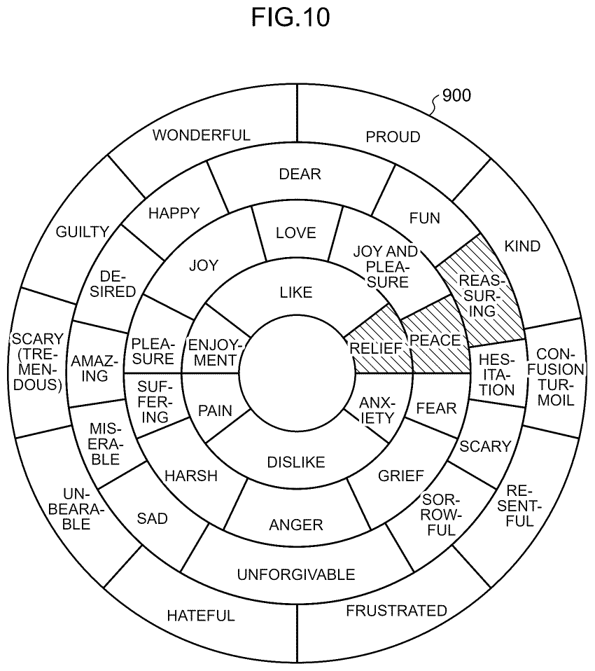

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Supplementary Note 1

A system comprising: a verbalization unit configured to verbalize spoken content using speech recognition technology; a response unit configured to generate a response based on the content verbalized by the verbalization unit and to respond by voice; an output unit configured to output the response generated by the response unit by voice; a provision unit configured to provide the voice output by the output unit to an elderly person; a monitoring unit configured to monitor the health condition of the elderly person based on the voice provided by the provision unit; and a notification unit configured to notify a medical institution or family in case of emergency based on the health condition monitored by the monitoring unit.

### Supplementary Note 2

The system according to Supplementary Note 1, wherein the monitoring unit is configured to periodically measure the heart rate or body temperature of the elderly person.

### Supplementary Note 3

The system according to Supplementary Note 1, wherein the notification unit is configured to notify a medical institution or family when an abnormality is detected.

### Supplementary Note 4

The system according to Supplementary Note 1, wherein the verbalization unit is configured to verbalize the spoken content of the elderly person using speech recognition technology.

### Supplementary Note 5

The system according to Supplementary Note 1, wherein the response unit is configured to generate a response based on the verbalized content.

### Supplementary Note 6

The system according to Supplementary Note 1, wherein the output unit is configured to output the response by voice.

### Supplementary Note 7

The system according to Supplementary Note 1, wherein the provision unit is configured to provide the voice to the elderly person.

### Supplementary Note 8

The system according to Supplementary Note 1, wherein the monitoring unit is configured to monitor the health condition.

### Supplementary Note 9

The system according to Supplementary Note 1, wherein the notification unit is configured to notify a medical institution or family in case of emergency.

### Supplementary Note 10

The system according to Supplementary Note 1, wherein the verbalization unit is configured to estimate emotion and adjust the verbalization expression method based on the estimated emotion.

### Supplementary Note 11

The system according to Supplementary Note 1, wherein the verbalization unit is configured to adjust the accuracy of verbalization according to the speaking speed or volume of the elderly person.

### Supplementary Note 12

The system according to Supplementary Note 1, wherein the verbalization unit is configured to improve the accuracy of verbalization by referring to the past conversation history of the elderly person.

### Supplementary Note 13

The system according to Supplementary Note 1, wherein the verbalization unit is configured to estimate the emotion of the elderly person and adjust the timing of verbalization based on the estimated emotion.

### Supplementary Note 14

The system according to Supplementary Note 1, wherein the verbalization unit is configured to apply region-specific speech recognition models to accommodate the dialects and accents of the elderly person.

### Supplementary Note 15

The system according to Supplementary Note 1, wherein the verbalization unit is configured to emphasize specific keywords in verbalization according to the spoken content of the elderly person.

### Supplementary Note 16

The system according to Supplementary Note 1, wherein the response unit is configured to estimate the emotion of the elderly person and adjust the tone and content of the response based on the estimated emotion.

### Supplementary Note 17

The system according to Supplementary Note 1, wherein the response unit is configured to generate a more personalized response by referring to the past conversation history of the elderly person.

### Supplementary Note 18

The system according to Supplementary Note 1, wherein the response unit is configured to generate a response including appropriate advice or cautions according to the health condition of the elderly person.

### Supplementary Note 19

The system according to Supplementary Note 1, wherein the response unit is configured to estimate the emotion of the elderly person and adjust the length of the response based on the estimated emotion.

### Supplementary Note 20

The system according to Supplementary Note 1, wherein the response unit is configured to generate a response including relevant information based on the hobbies or interests of the elderly person.

### Supplementary Note 21

The system according to Supplementary Note 1, wherein the response unit is configured to adjust the order or priority of responses according to the spoken content of the elderly person.

### Supplementary Note 22

The system according to Supplementary Note 1, wherein the output unit is configured to estimate the emotion of the elderly person and adjust the tone or speed of the output voice based on the estimated emotion.

### Supplementary Note 23

The system according to Supplementary Note 1, wherein the output unit is configured to adjust the volume or frequency of the output voice according to the hearing ability of the elderly person.

### Supplementary Note 24

The system according to Supplementary Note 1, wherein the output unit is configured to optimize the content of the output voice by referring to the past reactions of the elderly person.

### Supplementary Note 25

The system according to Supplementary Note 1, wherein the output unit is configured to estimate the emotion of the elderly person and adjust the timing of the output voice based on the estimated emotion.

### Supplementary Note 26

The system according to Supplementary Note 1, wherein the output unit is configured to detect environmental sounds of the elderly person and automatically adjust the volume of the output voice.

### Supplementary Note 27

The system according to Supplementary Note 1, wherein the output unit is configured to adjust the emphasized portions of the output voice according to the spoken content of the elderly person.

### Supplementary Note 28

The system according to Supplementary Note 1, wherein the provision unit is configured to estimate the emotion of the elderly person and adjust the content of the provided voice based on the estimated emotion.

### Supplementary Note 29

The system according to Supplementary Note 1, wherein the provision unit is configured to optimize the timing of the provided voice by referring to the past reactions of the elderly person.

### Supplementary Note 30

The system according to Supplementary Note 1, wherein the provision unit is configured to customize the content of the provided voice according to the health condition of the elderly person.

### Supplementary Note 31

The system according to Supplementary Note 1, wherein the provision unit is configured to estimate the emotion of the elderly person and adjust the order of the provided voice based on the estimated emotion.

### Supplementary Note 32

The system according to Supplementary Note 1, wherein the provision unit is configured to adjust the volume or tone of the provided voice according to the environment of the elderly person.

### Supplementary Note 33

The system according to Supplementary Note 1, wherein the provision unit is configured to adjust the level of detail of the provided voice according to the spoken content of the elderly person.

### Supplementary Note 34

The system according to Supplementary Note 1, wherein the monitoring unit is configured to estimate the emotion of the elderly person and adjust the frequency of monitoring based on the estimated emotion.

### Supplementary Note 35

The system according to Supplementary Note 1, wherein the monitoring unit is configured to improve the accuracy of monitoring by referring to the past health data of the elderly person.

### Supplementary Note 36

The system according to Supplementary Note 1, wherein the monitoring unit is configured to optimize the timing of monitoring according to the lifestyle rhythm of the elderly person.

### Supplementary Note 37

The system according to Supplementary Note 1, wherein the monitoring unit is configured to estimate the emotion of the elderly person and determine the priority of monitoring based on the estimated emotion.

### Supplementary Note 38

The system according to Supplementary Note 1, wherein the monitoring unit is configured to customize the content of monitoring by referring to the environmental data of the elderly person.

### Supplementary Note 39

The system according to Supplementary Note 1, wherein the monitoring unit is configured to adjust the items of monitoring according to the spoken content of the elderly person.

### Supplementary Note 40

The system according to Supplementary Note 1, wherein the notification unit is configured to estimate the emotion of the elderly person and adjust the content of notification based on the estimated emotion.

### Supplementary Note 41

The system according to Supplementary Note 1, wherein the notification unit is configured to improve the accuracy of notification by referring to the past health data of the elderly person.

### Supplementary Note 42

The system according to Supplementary Note 1, wherein the notification unit is configured to optimize the timing of notification according to the lifestyle rhythm of the elderly person.

### Supplementary Note 43

The system according to Supplementary Note 1, wherein the notification unit is configured to estimate the emotion of the elderly person and determine the priority of notification based on the estimated emotion.

### Supplementary Note 44

The system according to Supplementary Note 1, wherein the notification unit is configured to customize the content of notification by referring to the environmental data of the elderly person.

### Supplementary Note 45

The system according to Supplementary Note 1, wherein the notification unit is configured to adjust the items of notification according to the spoken content of the elderly person.

What is claimed is:

1. A system comprising:
circuitry configured to:
receive, from a client terminal, audio data captured by a microphone of the client terminal;
convert the audio data into text data by applying a trained neural network comprising at least one of a convolutional neural network, a recurrent neural network, or a Transformer-based model to acoustic feature vectors extracted from the audio data;
estimate an emotion of a user by applying an emotion identification model to at least one of the audio data or the text data to generate an emotion value;
generate, by inputting the text data and the emotion value into a data generation model obtained by deep learning on a neural network, inference data comprising response text;
convert the inference data into audio output data by applying a speech synthesis neural network to the response text;
transmit the audio output data to the client terminal for output via a speaker of the client terminal;
analyze time-series sensor data received from the client terminal using an anomaly detection model to generate an anomaly score indicating a degree of deviation from a reference range; and
transmit, when the anomaly score exceeds a threshold, an alert message to a designated terminal via a packet-switched network.

2. The system according to claim 1, wherein the time-series sensor data comprises at least one of heart rate data, body temperature data, or blood pressure data collected at periodic intervals from a biosensor of the client terminal.

3. The system according to claim 1, wherein the anomaly detection model comprises at least one of a long short-term memory network or a threshold judgment logic, and wherein the anomaly score is computed from a multidimensional time-series tensor comprising sensor measurements accumulated over a predetermined time window.

4. The system according to claim 1, wherein the circuitry is further configured to adjust at least one of a tone, a speed, or a content of the response text based on the emotion value, such that when the emotion value indicates sadness, the circuitry generates the response text using empathetic vocabulary, and when the emotion value indicates excitement, the circuitry generates the response text in a calm tone.

5. The system according to claim 1, wherein the acoustic feature vectors comprise at least one of mel spectrograms or mel-frequency cepstral coefficients extracted from the audio data sampled at a predetermined sampling rate.

6. The system according to claim 1, wherein the circuitry is further configured to adjust a recognition accuracy parameter of the trained neural network based on at least one of a speech rate or a volume level detected from the audio data.

7. The system according to claim 1, wherein the circuitry is further configured to retrieve, from a database, a dialogue history associated with the user, and to input the dialogue history as bias information into the data generation model to generate the inference data.

8. The system according to claim 1, wherein the speech synthesis neural network comprises at least one of a WaveNet model or a Tacotron model, and wherein the audio output data comprises a pulse-code modulation waveform generated at a predetermined sampling rate.

9. The system according to claim 1, wherein the circuitry is further configured to adjust at least one of a volume, a pitch, or a speech rate of the audio output data based on the emotion value estimated from the audio data.

10. The system according to claim 1, wherein the alert message comprises structured data indicating at least one of a time of anomaly occurrence, a measured value, a trend graph for a preceding time period, or a reason for anomaly judgment.

11. The system according to claim 1, wherein the circuitry is further configured to determine a priority of the alert message based on the anomaly score, such that when the anomaly score exceeds a first threshold, the circuitry transmits the alert message to a first designated terminal, and when the anomaly score exceeds a second threshold higher than the first threshold, the circuitry transmits the alert message to a second designated terminal.

12. The system according to claim 1, wherein the emotion identification model comprises a Transformer-based emotion classification model trained by supervised learning, and wherein the emotion value comprises an emotion label and an emotion score indicating a confidence level.

13. The system according to claim 1, wherein the circuitry is further configured to extract, from the text data, at least one keyword using a keyword extraction algorithm comprising at least one of TF-IDF or an attention mechanism, and to assign an emphasis instruction to the at least one keyword for the speech synthesis neural network.

14. The system according to claim 1, wherein the circuitry is further configured to detect an ambient noise level from environmental audio data received from the client terminal, and to adjust at least one of a volume or a frequency characteristic of the audio output data based on the ambient noise level.

15. The system according to claim 1, wherein the circuitry is further configured to store the time-series sensor data in a database as time-series vectors, and to apply at least one of moving average filtering or median filtering to the time-series vectors to remove noise before inputting the time-series vectors into the anomaly detection model.

16. The system according to claim 1, wherein the circuitry is further configured to receive motion event detection data from an acceleration sensor or a gyroscope sensor of the client terminal, and to trigger additional acquisition of the time-series sensor data in response to the motion event detection data.

17. The system according to claim 1, wherein the data generation model comprises at least one of a text generation AI, an image generation AI, or a multimodal generation AI, and wherein the data generation model is a fine-tuned model configured to output inference results from prompts without instructions.

18. A system comprising:
circuitry configured to:
receive, from a client terminal via a communication interface and a packet-switched network conforming to at least one of a 5G, Wi-Fi, or Bluetooth communication standard, audio data captured by a microphone of the client terminal;
extract acoustic feature vectors comprising at least one of mel spectrograms or mel-frequency cepstral coefficients from the audio data;
convert the audio data into text data by applying a trained neural network comprising at least one of a convolutional neural network, a recurrent neural network, or a Transformer-based speech recognition model to the acoustic feature vectors;
estimate an emotion of a user by applying an emotion identification model comprising a Transformer-based emotion classification model to at least one of the acoustic feature vectors or the text data, the emotion identification model outputting an emotion label and an emotion score;
retrieve, from a database, a dialogue history associated with the user;
generate, by inputting the text data, the emotion label, and the dialogue history into a data generation model obtained by deep learning on a neural network, inference data comprising response text, the response text being adjusted based on the emotion label;
convert the inference data into audio output data by applying a speech synthesis neural network comprising at least one of a WaveNet model or a Tacotron model to the response text, the audio output data comprising a pulse-code modulation waveform;
transmit the audio output data to the client terminal for output via a speaker of the client terminal;
analyze time-series sensor data received from the client terminal using an anomaly detection model comprising at least one of a long short-term memory network or a threshold judgment logic to generate an anomaly score, the time-series sensor data comprising at least one of heart rate data or body temperature data stored as time-series vectors; and
transmit, when the anomaly score exceeds a threshold, an alert message comprising structured data indicating at least one of a time of anomaly occurrence, a measured value, or a reason for anomaly judgment to a designated terminal via the packet-switched network.

19. The system according to claim 18, wherein the circuitry is further configured to determine a priority of the alert message based on the anomaly score, such that when the anomaly score exceeds a first threshold, the circuitry transmits the alert message to a first designated terminal, and when the anomaly score exceeds a second threshold higher than the first threshold, the circuitry transmits the alert message to a second designated terminal.

20. A method performed by circuitry of a system, the method comprising:
receiving, from a client terminal, audio data captured by a microphone of the client terminal;
converting the audio data into text data by applying a trained neural network comprising at least one of a convolutional neural network, a recurrent neural network, or a Transformer-based model to acoustic feature vectors extracted from the audio data;
estimating an emotion of a user by applying an emotion identification model to at least one of the audio data or the text data to generate an emotion value;
generating, by inputting the text data and the emotion value into a data generation model obtained by deep learning on a neural network, inference data comprising response text;
converting the inference data into audio output data by applying a speech synthesis neural network to the response text;
transmitting the audio output data to the client terminal for output via a speaker of the client terminal;
analyzing time-series sensor data received from the client terminal using an anomaly detection model to generate an anomaly score indicating a degree of deviation from a reference range; and
transmitting, when the anomaly score exceeds a threshold, an alert message to a designated terminal via a packet-switched network.
